(19) United States
(12) Patent Application Publication
(10) Pub. No.: US 2026/0365960 A1
(43) Pub. Date: Nov. 19, 2026

(54) DEVICES AND METHODS FOR MEMBRANE-DRIVEN GEOMETRIC CONTROL OF NANOPORE ELECTROPORATION FOR SPATIAL PATTERNING AND REDOX-MEDIATED BUFFERS FOR CELL ELECTROPORATION

(71) Applicant: The Regents of The University of Michigan, Ann Arbor, MI (US)

(72) Inventors: Tianxiang LIU, Superior Township, MI (US); Matthew Lee MANION, Ann Arbor, MI (US); Emily MCCORKLE, Ann Arbor, MI (US); Sasha Cai LESHER-PEREZ, Ann Arbor, MI (US)

(73) Assignee: The Regents of The University of Michigan, Ann Arbor, MI (US)

(21) Appl. No.: 19/680,219

(22) Filed: May 18, 2026

Related U.S. Application Data
(60) Provisional application No. 63/807,290, filed on May 16, 2025.
(60) Provisional application No. 63/810,471, filed on May 22, 2025.

(51) Int. Cl. C12M 1/42; C12M 1/12

(52) U.S. Cl. CPC C12M 35/02; C12M 25/02

(57) ABSTRACT

A cell manipulation device has first and second electrodes and an insulating porous membrane supporting cells. The membrane has pores (e.g., nanopores) and may define an active area having a cross-sectional shape with a perimeter-to-area ratio of ≥about 1 or a rectilinear pattern defined by an electrically non-conductive material. At least one cargo material is delivered to at least one cell on the insulating porous membrane when a potential is applied to the first and second electrodes. An electroporation fluid composition is also provided including a redox active compound, at least one cargo material to be delivered to or removed from at least one cell when a potential is applied to the electroporation fluid, and a carrier liquid. Methods of manipulating cells are also provided in such cell manipulation devices or with such electroporation fluid compositions via electroporation processes.

### CROSS-REFERENCE TO RELATED APPLICATIONS

This application claims the benefit of U.S. Provisional Application No. 63/807,290 filed May 16, 2025 and U.S. Provisional Application No. 63/810,471 filed May 22, 2025. The entire disclosures of each of the above applications are incorporated herein by reference.

### GOVERNMENT SUPPORT

This invention was made with government support under 2243104 awarded by the National Science Foundation. The government has certain rights in the invention.

### FIELD

The present disclosure relates to a cell manipulation device or system, which can provide membrane-driven geometric control of nanopore electroporation for spatial patterning, as well as methods of manipulating cells in such a device or system. In other aspects, the present disclosure relates to an electroporation fluid composition for an electroporation or nanopore electroporation process of at least one cell, as well as methods of manipulating at least one cell via such a process in the presence of the electroporation fluid composition comprising a redox active compound.

### BACKGROUND

This section provides background information related to the present disclosure which is not necessarily prior art.

The efficient introduction of therapeutic cargo or payloads, such as macromolecules (e.g., biomolecules like DNA and proteins), directly into multiple cells at once to manipulate them is integral for multiple biological tools and medical related applications, including cellular and genetic engineering, genetic therapies, cell imaging, and biophysical models. One effective method of introducing macromolecules is through electroporation, in which cell membranes are disrupted through an externally applied voltage, so that “electropores” in their phospholipid membrane are generated across the cell and macromolecules can diffuse into the cell before the pores close. However, traditional electroporation can be detrimental to cell health and function, because the non-specific disruption of the cell membrane in all directions causes damage and potentially cell death. High voltage pulses (e.g., greater than about 500 V/mm) can cause cell death through a variety of mechanisms. Further, the transport into the cell is diffusion limited and causes low efficiency delivery.

Recently, the use of a nanoporous membrane having a plurality of nanopores disposed between electrodes has been used to localize the electroporation of cells cultured on top of this membrane. It utilizes an electrically insulating membrane with cylindrical nanochannels whose size, shape, and distribution determine the membrane's local electric field. This membrane sits on the electrode and localizes the applied voltage to permeate the phospholipid bilayer membranes of cells growing on it, resulting in temporary pores forming across cellular membranes. Cells with pores can readily uptake delivered genetic material or drugs, among other cargo, from the surrounding medium. Once the electrical field is stopped, the pores reseal, leaving the transfected cells viable. In certain aspects, the insulating membrane may be a nanoporous polycarbonate track-etched (PCTE) membrane.

In nanopore/nanochannel electroporation (NanoEP), the pores only occur at the nanopore-cell junction directly above the membrane, where the externally applied electric field is concentrated and magnified, limiting the disruption of the cell membrane and better preserving cell viability. Cells with pores can readily uptake delivered genetic material or drugs, among other cargo, from the surrounding medium. Further, the macromolecular cargo can be accelerated in a charge-specific manner inside vertically aligned nanopores, determined by the polarity of the external electric field, and be effectively “injected” into the cells. This bypasses the slow diffusion process in traditional electroporation to greatly enhance the delivery efficiency and specificity of desired macromolecular cargos. This nanochannel electroporation (NanoEP) offers a gentler, scalable, and more precise alternative for cell cargo manipulations. It would be desirable to develop new systems and methods for cell manipulation based on nanochannel electroporation with enhanced control over delivery of cargo materials to cells.

Further, while performing either bulk EP or NanoEP, the electrodes may facilitate chemical reactions that can negatively affect cell health or hinder cargo manipulation. These reactions can cause pH changes, free radical generation, electrode ion leaching, and gas bubble formation. This unfavorable electrochemical environment can trigger cell death. Thus, it would be desirable to develop new systems and methods for cell manipulation based on nanochannel electroporation with enhanced safety and minimal harm to cells during and after delivery of cargo materials when exposed to an applied electrical potential or voltage.

### SUMMARY

This section provides a general summary of the disclosure, and is not a comprehensive disclosure of its full scope or all of its features.

In certain aspects, the present disclosure relates to a cell manipulation device. The cell manipulation device may comprise a first electrode, a second electrode, and an insulating porous membrane configured to support a plurality of cells. The insulating porous membrane comprises a plurality of pores and defines a cross-sectional shape having a perimeter-to-area ratio of greater than or equal to about 0.75. The cell manipulation device also comprises at least one cargo material to be delivered to at least a portion of the plurality of cells disposed on the insulating porous membrane through at least one pore of the plurality of pores when a potential is applied to the first electrode and the second electrode.

In one aspect, the device further comprises a receptacle disposed adjacent to the insulating porous membrane that is electrically non-conductive and configured to receive an electroporation fluid comprising the at least one cargo material.

In one further aspect, the receptacle defines an opening having the cross-sectional shape and defining the active region through which the electroporation fluid comprising the at least one cargo material can pass.

In one aspect, the perimeter-to-area ratio is greater than or equal to about 2.

In one aspect, the cross-sectional shape defines at least two sides.

In one further aspect, the cross-sectional shape is selected from the group consisting of: a polygon, a triangle, a rectangle, a square, a quadrilateral, a pentagon, a hexagon, a serpentine shape, and combinations thereof.

In one aspect, the cross-sectional shape comprises a serpentine shape.

In one aspect, the at least one cargo material is a biomolecule.

In one aspect, the at least one cargo material comprises a biomaterial selected from the group consisting of: a pharmaceutical active ingredient, a protein, a peptide, a growth factor, a biofactor, a cytokine, a hormone, a gene therapy material, a cell therapy material, a nanomaterial, an imaging agent, and combinations thereof.

In one further aspect, the biomaterial comprises a genetic therapy agent comprising a polynucleotide agent selected from the group consisting of: DNA, RNA, plasmids, plasmid DNA (pDNA), short interfering sequence of double stranded RNA (siRNA), messenger RNA (mRNA), transfer RNA, ribosomal RNA, small nuclear RNA, single stranded DNA, microDNA, guide RNA, CRISPR CAS-9, aptamers, and combinations thereof.

In one aspect, the insulating porous membrane comprises polycarbonate.

In further aspects, the present disclosure relates to a cell manipulation device. The device includes a first electrode, a second electrode, and an insulating porous membrane configured to support a plurality of cells. The insulating porous membrane comprises a plurality of pores. Further, the insulating porous membrane defines a pattern of electrically non-conductive material disposed on the surface to define an active region. At least one cargo material is provided to be delivered to at least a portion of the plurality of cells disposed on the insulating porous membrane through at least one pore of the plurality of pores in the active region when a potential is applied to the first electrode and the second electrode.

In one aspect, the electrically non-conductive material comprises a polymeric material selected from the group consisting of: polydimethylsiloxane (PDMS), an epoxy-based photoresist, and combinations thereof.

In one aspect, the pattern defines an array comprising a plurality of shapes selected from the group consisting of: a dot, a circle, a polygon, a triangle, a rectangle, a square, a quadrilateral, a pentagon, a hexagon, a serpentine shape, and combinations thereof.

In one aspect, the pattern is a rectilinear pattern.

In one aspect, the pattern defines at least one continuous or discontinuous shape selected from the group consisting of: a polygon, a triangle, a rectangle, a square, a quadrilateral, a pentagon, a hexagon, a serpentine shape, and combinations thereof.

In one aspect, the device further includes a receptacle disposed adjacent to the insulating porous membrane that is electrically non-conductive and configured to receive an electroporation fluid comprising the at least one cargo material.

In one aspect, the receptacle defines an opening having the cross-sectional shape and defining the active region through which the electroporation fluid comprising the at least one cargo material can pass.

In one aspect, the at least one cargo material is a biomolecule.

In one aspect, the at least one cargo material comprises a biomaterial selected from the group consisting of: a pharmaceutical active ingredient, a protein, a peptide, a growth factor, a biofactor, a cytokine, a hormone, a gene therapy material, a cell therapy material, a nanomaterial, an imaging agent, and combinations thereof.

In one further aspect, the biomaterial comprises a genetic therapy agent comprising a polynucleotide agent selected from the group consisting of: DNA, RNA, plasmids, plasmid DNA (pDNA), short interfering sequence of double stranded RNA (siRNA), messenger RNA (mRNA), transfer RNA, ribosomal RNA, small nuclear RNA, single stranded DNA, microDNA, guide RNA, CRISPR CAS-9, aptamers, and combinations thereof.

In one aspect, the insulating porous membrane comprises polycarbonate.

In yet other aspects, the present disclosure relates to a method of manipulating cells. The method comprises introducing at least one cargo material into at least one cell supported on an insulating porous membrane when an electric field is generated between a first electrode and a second electrode in a nanopore electroporation process. The insulating porous membrane comprises a plurality of nanopores and defines a cross-sectional shape having a perimeter-to-area ratio of greater than or equal to about 0.75.

In one aspect, the at least one cell comprises a plurality of cells defining a monolayer on the insulating porous membrane.

In one aspect, the at least one cell is a plurality of cells supported on the insulating porous membrane and the introducing of the at least one cargo material is a first amount for a first cell of the plurality of cells and a second amount for a second cell of the plurality of cells, wherein the first amount is distinct from the second amount.

In one aspect, the perimeter-to-area ratio is greater than or equal to about 2.

In one aspect, the cross-sectional shape defines at least two sides.

In one aspect, the cross-sectional shape is selected from the group consisting of: a polygon, a triangle, a rectangle, a square, a quadrilateral, a pentagon, a hexagon, a serpentine shape, and combinations thereof.

In one aspect, the cross-sectional shape comprises a serpentine shape.

In one aspect, the at least one cargo material is a biomolecule.

In one aspect, the at least one cargo material comprises a biomaterial selected from the group consisting of: a pharmaceutical active ingredient, a protein, a peptide, a growth factor, a biofactor, a cytokine, a hormone, a gene therapy material, a cell therapy material, a nanomaterial, an imaging agent, and combinations thereof.

In one further aspect, the biomaterial comprises a genetic therapy agent comprising a polynucleotide agent selected from the group consisting of: DNA, RNA, plasmids, plasmid DNA (pDNA), short interfering sequence of double stranded RNA (siRNA), messenger RNA (mRNA), transfer RNA, ribosomal RNA, small nuclear RNA, single stranded DNA, microDNA, guide RNA, CRISPR CAS-9, aptamers, and combinations thereof.

In certain other aspects, the present disclosure relates to a cell manipulation device. The cell manipulation device may comprise a first electrode, a second electrode, and an insulating porous membrane configured to support a plurality of cells. The insulating porous membrane comprises a plurality of pores and defines a cross-sectional serpentine shape. The serpentine shape may have at least two 90° turns. The serpentine shape may have a perimeter-to-area ratio of greater than or equal to about 2. The cell manipulation device also comprises at least one cargo material to be delivered to at least a portion of the plurality of cells disposed on the insulating porous membrane through at least one pore of the plurality of pores when a potential is applied to the first electrode and the second electrode.

In one aspect, the device further comprises a receptacle disposed adjacent to the insulating porous membrane that is electrically non-conductive and configured to receive an electroporation fluid comprising the at least one cargo material.

In one further aspect, the receptacle defines an opening having the cross-sectional shape and defining the active region through which the electroporation fluid comprising the at least one cargo material can pass.

In one aspect, the at least one cargo material is a biomolecule.

In one aspect, the at least one cargo material comprises a biomaterial selected from the group consisting of: a pharmaceutical active ingredient, a protein, a peptide, a growth factor, a biofactor, a cytokine, a hormone, a gene therapy material, a cell therapy material, a nanomaterial, an imaging agent, and combinations thereof.

In one further aspect, the biomaterial comprises a genetic therapy agent comprising a polynucleotide agent selected from the group consisting of: DNA, RNA, plasmids, plasmid DNA (pDNA), short interfering sequence of double stranded RNA (siRNA), messenger RNA (mRNA), transfer RNA, ribosomal RNA, small nuclear RNA, single stranded DNA, microDNA, guide RNA, CRISPR CAS-9, aptamers, and combinations thereof.

In one aspect, the insulating porous membrane comprises polycarbonate.

The present disclosure further contemplates method of manipulating cells that includes introducing at least one cargo material into at least one cell supported on a surface of an insulating porous membrane when an electric field is generated between a first electrode and a second electrode in a nanopore electroporation process. The insulating porous membrane comprises a plurality of nanopores. Further, on its surface, the insulating porous membrane has a pattern of electrically non-conductive material disposed thereon that defines an active region through which the at least one cargo material passes into the at least one cell.

In one aspect, the electrically non-conductive material comprises a polymeric material selected from the group consisting of: polydimethylsiloxane (PDMS), an epoxy-based photoresist, and combinations thereof.

In one aspect, the pattern is a rectilinear pattern.

In one aspect, the pattern defines an array comprising a plurality of shapes selected from the group consisting of: a dot, a circle, a polygon, a triangle, a rectangle, a square, a quadrilateral, a pentagon, a hexagon, a serpentine shape, and combinations thereof.

In one aspect, the pattern defines at least one continuous or discontinuous shape selected from the group consisting of: a polygon, a triangle, a rectangle, a square, a quadrilateral, a pentagon, a hexagon, a serpentine shape, and combinations thereof.

In one aspect, the method further comprises a receptacle disposed adjacent to the insulating porous membrane that is electrically non-conductive and that receives an electroporation fluid comprising the at least one cargo material.

In one aspect, the receptacle defines an opening having the cross-sectional shape and defines the active region through which the electroporation fluid comprising the at least one cargo material passes.

In one aspect, the at least one cell comprises a plurality of cells defining a monolayer on the insulating porous membrane.

In one aspect, the at least one cell is a plurality of cells supported on the insulating porous membrane and the introducing of the at least one cargo material is a first amount for a first cell of the plurality of cells and a second amount for a second cell of the plurality of cells, wherein the first amount is distinct from the second amount.

In one aspect, the at least one cargo material is a biomolecule.

In one aspect, the at least one cargo material comprises a biomaterial selected from the group consisting of: a pharmaceutical active ingredient, a protein, a peptide, a growth factor, a biofactor, a cytokine, a hormone, a gene therapy material, a cell therapy material, a nanomaterial, an imaging agent, and combinations thereof.

In one aspect, the biomaterial comprises a genetic therapy agent comprising a polynucleotide agent selected from the group consisting of: DNA, RNA, plasmids, plasmid DNA (pDNA), short interfering sequence of double stranded RNA (siRNA), messenger RNA (mRNA), transfer RNA, ribosomal RNA, small nuclear RNA, single stranded DNA, microDNA, guide RNA, CRISPR CAS-9, aptamers, and combinations thereof.

In certain further aspects, the present disclosure relates to an electroporation fluid composition for an electroporation process for at least one cell. The electroporation fluid composition comprises a redox active compound, at least one cargo material to be delivered to or removed from the at least one cell when a potential is applied to the electroporation fluid, and a carrier liquid.

In one aspect, the redox active compound comprises potassium ferrocyanide, potassium ferricyanide, ferrocenemethanol, and combinations thereof.

In one aspect, the redox active compound comprises potassium ferrocyanide and potassium ferricyanide.

In one further aspect, the potassium ferrocyanide is present at a concentration of greater than or equal to about 1 mM to less than or equal to about 10 mM in the electroporation fluid composition and the potassium ferricyanide is present at a concentration of greater than or equal to about 1 mM to less than or equal to about 10 mM in the electroporation fluid composition.

In one further aspect, the redox active compound further comprises ferrocenemethanol.

In one further aspect, the ferrocenemethanol is present at a concentration of greater than or equal to about 1 mM to less than or equal to about 50 mM in the electroporation fluid composition.

In one aspect, a total amount of the redox active compound is present at a concentration of greater than or equal to about 1 mM to less than or equal to about 70 mM in the electroporation fluid composition.

In one aspect, the carrier liquid comprises water.

In one aspect, the electroporation fluid composition further comprises at least one component selected from the group consisting of: a salt, a sugar, a buffering agent, a chelating agent, a pH adjusting agent, adenosine triphosphate (ATP), and combinations thereof.

In one aspect, the at least one cargo material comprises a biomaterial selected from the group consisting of: a pharmaceutical active ingredient, a protein, a peptide, a growth factor, a biofactor, a cytokine, a hormone, a gene therapy material, a cell therapy material, a nanomaterial, an imaging agent, and combinations thereof.

In one aspect, the biomaterial comprises a genetic therapy agent comprising a polynucleotide agent selected from the group consisting of: DNA, RNA, plasmids, plasmid DNA (pDNA), short interfering sequence of double stranded RNA (siRNA), messenger RNA (mRNA), transfer RNA, ribosomal RNA, small nuclear RNA, single stranded DNA, microDNA, guide RNA, CRISPR CAS-9, aptamers, and combinations thereof.

In certain other aspects, the present disclosure relates to a method of manipulating cells. The method optionally comprises introducing or removing at least one cargo material into or from at least one cell supported on an insulating porous membrane when an electric field is generated between a first electrode and a second electrode in either an electroporation or a nanopore electroporation process. The at least one cell is in contact with an electroporation fluid composition. The electroporation fluid composition may comprise a redox active compound, at least one cargo material, and a carrier liquid.

In one aspect, the redox active compound comprises potassium ferrocyanide, potassium ferricyanide, ferrocenemethanol, and combinations thereof.

In one aspect, the redox active compound comprises potassium ferrocyanide and potassium ferricyanide.

In one further aspect, the potassium ferrocyanide is present at a concentration of greater than or equal to about 1 mM to less than or equal to about 10 mM in the electroporation fluid composition and the potassium ferricyanide is present at a concentration of greater than or equal to about 1 mM to less than or equal to about 10 mM in the electroporation fluid composition.

In one further aspect, the redox active compound further comprises ferrocenemethanol.

In one further aspect, the ferrocenemethanol is present at a concentration of greater than or equal to about 1 mM to less than or equal to about 50 mM in the electroporation fluid composition.

In one aspect, a total amount of the redox active compound is present at a concentration of greater than or equal to about 1 mM to less than or equal to about 70 mM in the electroporation fluid composition.

In one aspect, the carrier liquid comprises water.

In one aspect, the electroporation fluid composition further comprises at least one component selected from the group consisting of: a salt, a sugar, a buffering agent, a chelating agent, a pH adjusting agent, adenosine triphosphate (ATP), and combinations thereof.

In one aspect, the at least one cargo material comprises a biomaterial selected from the group consisting of: a pharmaceutical active ingredient, a protein, a peptide, a growth factor, a biofactor, a cytokine, a hormone, a gene therapy material, a cell therapy material, a nanomaterial, an imaging agent, and combinations thereof.

In one aspect, the biomaterial comprises a genetic therapy agent comprising a polynucleotide agent selected from the group consisting of: DNA, RNA, plasmids, plasmid DNA (pDNA), short interfering sequence of double stranded RNA (siRNA), messenger RNA (mRNA), transfer RNA, ribosomal RNA, small nuclear RNA, single stranded DNA, microDNA, guide RNA, CRISPR CAS-9, aptamers, and combinations thereof.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A-1F. FIG. 1A shows two side views of a device set-up for cargo delivery and depletion experiments for nano-pore electroporation. FIG. 1B shows BSA Alexa Fluor™ 488 Delivery into HT-1080 cells (blue-Hoechst); scale bar is 170 micrometers (left) scale bar is 430 micrometers (right). FIG. 1C shows a heat map of rate constants in 2 mm device for calcein depletion; scale bar is 500 micrometers. FIG. 1D shows a 6 mm circle membrane with 6 bins where center to edge is R1-R6 (left), normalized intensity of each bin over time for 6 mm device at 15 V, 20 Hz frequency, 1 ms pulse width, for 120s (middle), normalized rate constant for reach bin showing increase in rate constant from R1 to R6 (right). FIG. 1E shows normalized rate constants from experimental data across binned regions (points) compared with simulated transport rates across applied voltages (line), capturing the nonlinear dependence of cargo transport on voltage drop. FIG. 1F shows a schematic cross-sectional illustration of lateral voltage distribution across a PCTE nanoporous insulating membrane during cargo delivery (top) and depletion (bottom), illustrating enhanced cargo flux at the membrane edge.

FIGS. 2A-2F. FIG. 2A shows a schematic illustration of a device setup for cathodic ITO reduction experiments on the left. On the right top: a pristine ITO film before electroporation (left side, scale bar 500 nm) and a reduced ITO film after electroporation (right side, scale bar 1 micrometer) are shown. On the bottom right: a bright field optical micrograph showing darkened cathode following electric stimulation of 2 mm device with PCTE membrane (scale bar, 500 μm). FIG. 2B shows transmitted light intensity decreases over time across six (6) concentric radial bins of a 2 mm circular PCTE membrane device over time (30 V, 20 Hz, 1 ms square-wave pulse width, 40 s duration; scale bar, 500 μm). FIG. 2C shows COMSOL-simulated lateral distribution of vertical voltage drop across the electrolyte in a 6 mm circular device with no PCTE membrane under 30 V cathode-to-anode bias. FIG. 2D shows a heat map of ITO reduction rate constants in a 6 mm circular device with no PCTE membrane under 30 V cathode-to-anode bias (30 V, 20 Hz, 1 ms square-wave pulse width, 40 s duration). FIG. 2E shows a bright field optical micrograph showing the darkened cathode under 30 V cathode-to-anode bias (same device as in FIG. 2D). FIG. 2F shows a heat map of normalized depletion rate constants predicted using the modified Nernst-Planck model. The line profiles above panels in FIGS. 2C-2F represent cross-sections through each 2D heat map, taken along an approximate 6 mm slice indicated on the corresponding map.

FIGS. 3A-3J. FIG. 3A shows a schematic illustration of NanoEP device setup and imaging orientation for electrophoretically driven cargo transport. FIG. 3B shows full-device fluorescence micrographs of Calcein AM-stained HT-1080 cells in triangular) (60°, square (90°), and obtuse (120°) geometries (scale bars, 1 mm). FIG. 3C shows normalized model rate constant heat maps for 60°, 90°, and 120° NanoEP devices. FIG. 3D shows representative experimental rate constant heat maps of calcein depletion for 60°, 90°, 120° NanoEP devices (scale bars, 430 μm). FIG. 3E shows a comparison of simulated and experimental normalized rate constants as a function of distance from corner for 60° NanoEP devices (n=3; scale bar, 430 μm). FIG. 3F shows experimental Δkₙₒᵣₘ (left) and characteristic length (right) for each device geometry tested (n=3). Significance for each metric was evaluated using a 1-way ANOVA with post hoc Tukey test. FIG. 3G shows spatial mean percent error between model and experiment for each geometry, where each point i is a distance coordinate from the device corner. FIG. 3H shows fluorescence intensity profiles of BSA-AF647 (2.5 mg/mL) delivery as a function of distance from a 60° device corner under 20 V, 20 Hz, 1 ms square-wave pulse width, 10 s duration (n=3; scale bar, 430 μm). FIG. 3I shows schematic illustration of NanoEP device setup and imaging orientation for anti-electrophoretically driven cargo transport. FIG. 3J shows average calcein depletion rate constants under electrophoretic versus anti-electrophoretic device configurations (n=3 per device geometry). Significance was evaluated using Welch's two sample t-test.

FIGS. 4A-4K. FIG. 4A shows a full-device fluorescent micrograph of Calcein AM-stained HT-1080 cells in a serpentine device geometry on the left (scale bar, 1 mm). On the right, rate constant heat maps of calcein depletion in circular (top) versus serpentine (bottom) geometries at 15 V, 20 Hz, 1 ms square-wave pulses, 120 s duration (scale bars, 430 μm). FIG. 4B shows a comparison of depletion rate constant as a function of distance from an edge for serpentine versus circular devices (n=3 per geometry) for serpentine and circle for individual devices. FIG. 4C shows on the left: a full-device fluorescent micrograph of a serpentine device following BSA-AF647 delivery (2.5 mg/mL; scale bar, 1 mm). On the right, FIG. 4C shows representative fluorescent micrographs for BSA-AF647 delivery (2.5 mg/mL) in circular (top) and serpentine (bottom) devices at 20 V, 20 Hz, 1 ms square-wave pulse width, 10 s duration (n=3 per geometry; scale bars, 200 μm). FIG. 4D shows a histogram of single-cell BSA-AF647 fluorescence intensities in circular versus serpentine geometries. FIG. 4E shows on the left: full-device fluorescent micrograph of a serpentine device following plasmid (pLenti3.7-DsRed, molecular weight of about 5 MDa, 100 ng/μL, YOYO-1 labeled) delivery (scale bar, 1 mm). FIG. 4E on the right shows representative fluorescent micrographs in circular (top) versus serpentine (bottom) devices at 25 V, 1 Hz, 10 ms square-wave pulse width, 4 s duration (scale bars, 200 μm). FIG. 4F shows a histogram of single-cell plasmid+YOYO-1 fluorescence intensities per cell in circular versus serpentine geometries. FIG. 4G shows on the left: a full-device fluorescent micrograph of a serpentine device showing DsRed protein expression 48 hours after plasmid delivery (pLenti3.7-DsRed, 5 ng/μL; scale bar, 1 mm). FIG. 4G shows on the right: representative fluorescent micrographs for DsRed expression in circular (top) versus serpentine (bottom) devices at 25 V, 1 Hz, 10 ms square-wave pulse width, 4 seconds duration (scale bars, 200 μm). FIG. 4H shows histogram of single-cell DsRed fluorescence intensities in circular versus serpentine geometries. FIG. 4I shows a summary of fold-changes in fluorescent intensity per cell for BSA-AF647, plasmid+YOYO-1, and DsRed expression in circular versus serpentine geometries. Significance for each pair was evaluated using Welch's two-sample t-test. FIG. 4J shows an average depletion rate constant for electrophoretically versus anti-electrophoretically driven cargo transport in circular versus serpentine geometries. Significance was evaluated using Welch's two-sample t-test. FIG. 4K shows cell viability 24 h (100 ng/μL samples) and 48 h (5 ng/μL and control samples) post-transfection for plasmid+YOYO-1 delivery experiments.

FIG. 5 shows perimeter-to-area ratios for different membrane active area shapes, including a circle (perimeter-to-area ratio of 0.67), square (perimeter-to-area ratio of 0.75), triangle (perimeter-to-area ratio of 0.87), and a serpentine pattern (perimeter-to-area ratio of 2.03).

FIGS. 6A-6E. FIG. 6A shows a sticker with pillars formed of an electrically non-conductive material attached to a glass slide to raise the membrane with respect to an adjacent electrode in a cell manipulation device for nanoporation. FIG. 6B show the sticker being removed so only the pillars remain. FIG. 6C shows pillars are then plasma bonded or attached to the NanoEP devices. FIG. 6D shows BSA-AF647 localization from folds of the PCTE membrane with no pillars underneath the membrane (scale bar, 1 mm). FIG. 6E shows BSA-AF647 spreading with 200 μm tall pillars underneath membrane (scale bar, 1 mm).

FIGS. 7A-7J. FIG. 7A shows nanoEP-mediated GFP depletion from NIH/3T3 cells at 15 V, 20 Hz, 1 ms square-wave pulses for 120 s in circular (top) and serpentine (bottom) geometries (scale bars, 430 μm). FIG. 7B shows comparison of GFP depletion rate constants in circular vs. serpentine devices (n=4 per geometry). FIG. 7C shows BSA-AF488 delivery (2.5 mg/mL) at 20 V, 20 Hz, 1 ms square-wave pulses, 10 s duration in three cell types: MDA-MB-231 (top), HCE-S (middle), and IM-HA (bottom), shown in circular (left) and serpentine (right) geometries (scale bar, 300 μm). FIG. 7D shows a summary of fold changes in single-cell BSA-AF488 fluorescent intensity in serpentine versus circular geometries for MDA-MB-231, HCE-S, and IM-HA. Significance for each pair was evaluated using Welch's two-sample t-test. FIG. 7E shows BSA-AF488 delivery (2.5 mg/mL) at 20 V, 20 Hz, 1 ms square-wave pulses, 10 s duration in circular devices of six diameters: 2, 3, 6 mm (scale bar, 1 mm), 8.5, 13.4 and 19 mm (scale bar, 2 mm). FIG. 7F shows mean BSA-AF488 fluorescence intensity as a function of device diameter (n=3-4 per device diameter). FIG. 7G shows BSA-AF488 delivery (2.5 mg/mL) at 20 V, 20 Hz, 1 ms square-wave pulses, 10 s duration in scaled serpentine devices with 1×(scale bar, 2 mm) 2×(scale bar, 5 mm), and 5×(scale bar, 5 mm) surface areas. FIG. 7H shows a summary of fold changes in single-cell BSA-AF488 fluorescent intensity in serpentine versus circular geometries at 1 time, 2 times, and 5 times surface areas. Significance for each pair was evaluated using Welch's two-sample t-test. FIG. 7I on the top shows: BSA-AF488 delivery (2.5 mg/mL) at 20 V, 20 Hz, 1 ms square-wave pulses, 10 s duration in circular devices and 5 times serpentine device. FIG. 7I on the bottom shows: overlay with Hoechst and BSA-AF488 in circular and 5 times serpentine device, scale bars, 300 μm). FIG. 7J shows delivery efficiency versus cell throughput, where cell throughput is cell seeding density multiplied by cell seeding area. Literature values shown are average values at their self-reported optimum for several adherent cell types. Nanopore, EP (Circular) contains the 2 mm-13.4 mm diameter circular data, and Nanopore, EP (Serpentine) contains the 5× serpentine data. The experimental work is reports average delivery efficiency from BSA-AF488 delivery.

FIGS. 8A-8E show the 3D models for various membrane-shaped devices along with dimensions for each active region feature. FIG. 8A shows a two-layer click-in mold design for triangular-shaped membranes. FIG. 8B shows a two-layer click-in mold design for square-shaped membrane devices. FIG. 8C shows a two-layer click-in mold design for obtuse-shaped (e.g., circular sector shaped) devices. FIG. 8D shows two-layer click-in mold design for serpentine-shaped membrane devices. Finally, FIG. 8E shows a single layer mold for conventional circular-shaped devices.

FIG. 9 shows an electroporation or nanopore/nanochannel electroporation device for manipulating cells, including a lumped model showing system parameters.

FIG. 10 shows a simulation of a nanopore electroporation (NanoEP) device geometry, comparing an electrolyte voltage drop (electromotive driving force) over different charge transfer resistances and cell configurations. On the left, the cells either cover the entire nanochannel membrane or there was unequal cell coverage with a gap in the middle (as shown on the right).

FIGS. 11A-11C takes simulation geometry from FIG. 10 (right side) to simulate transport rates of charged molecules out of cells on a membrane. FIG. 11A shows high resistance conditions, while FIG. 11B shows low resistance conditions that compare fraction of particles in cells near a center of the membrane versus cells near an edge of the membrane. FIG. 11C shows a schematic of cells on a nanoporous membrane.

FIG. 12 shows experimental impedance data collected for a commercial electroporation buffer (EP Buffer) and an inventive electroporation buffer with equimolar potassium ferrocyanide and potassium ferricyanide (FeCN Buffer) prepared in accordance with certain aspects of the present disclosure, both pre-electroporation (pre-EP) and post-electroporation (post-EP).

FIGS. 13A-13C show experimental electroporation data collected using the test device pictured in FIG. 13A. FIG. 13B shows fluorescent intensity change data for 5 mM FeCN electroporation fluid/buffer prepared in accordance with certain aspects of the present disclosure, the commercial electroporation (EP) buffer, and a comparative commercial EP buffer with 15 mM potassium chloride. In FIG. 13C, simulations of the expected transport out of the cells as a function of the R_{CT} and buffer conductivity were compared to experimental results, which align well with the simulated predictions.

FIGS. 14A-14B show HT-1080 (human fibrosarcoma) cell viability data collected using an automated imaging microscope with a cell incubator (37 °C, 5% CO₂) on the left in FIG. 14A. In FIG. 14B, HT-1080 cell viability data collected 24 hours post-electroporation is shown for cells having undergone nanopore electroporation with 5 mM FeCN electroporation fluid/buffer prepared in accordance with certain aspects of the present disclosure as compared to cells in the commercial electroporation (EP) buffer.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

In various aspects, the present disclosure contemplates a cell manipulation system or device, which may be a microfluidic device. Generally, a microfluidic device has at least one feature that is on a microscale, so that it is configured to process fluids in a microfluidic device. As used herein, “microscale” refers to a structure having at least one dimension that is less than or equal to about 1,000 micrometers (μm) and optionally less than or equal to about 500 micrometers (μm). As used herein, reference to a microscale, microfluidic, microchannel, microfluidic channel, or a microstructure, encompasses smaller structures, such as those being on a nanoscale.

The cell manipulation system is configured to conduct an electroporation transfer process (e.g., a nanoporation process), where at least one cargo material is to be delivered (e.g., from an electroporation fluid composition) to at least one cell through the at least one opening/pore on an insulating porous membrane and through at least one temporary opening in the cell membrane created when a potential is applied/generated by electrodes of opposite polarity. Thus, the system includes a first electrode having a first polarity and a second electrode having a second opposite polarity. The first electrode and the second electrode are each formed of an electrically conductive material and are arranged in a spaced-apart relationship from one another to define a distance therebetween. The first and second electrode may comprise the same or distinct electrically conductive materials used to form electrodes, including by way of non-limiting example, indium tin oxide (ITO), gold (Au), silver (Ag), aluminum (Al), copper (Cu), alloys thereof, or any other electrically conductive material known to those of skill in the art.

The cell manipulation device further includes a porous membrane disposed between the first electrode and the second electrode. The porous membrane may be formed of an electrically non-conductive and insulating material, thus referred to herein as an insulating membrane or insulating porous membrane. The insulating porous membrane is configured to support and promote growth of cells. As will be described further herein, the membrane comprises a plurality of pores, which may have a round or circular cross-sectional shape, thus forming cylindrical pores or channels, although other pore geometries are also contemplated. In certain aspects, the present disclosure contemplates membrane-driven geometric control of nanopore electroporation for spatial patterning by using geometric control via geometry, such as sharp angles, to alter the electric field strength in certain locations to allow for varying amounts of a cargo material to be delivered into cells (e.g., a monolayer of cells disposed on the insulating porous membrane). In this way, a gradient in expression of the same gene can be achieved in certain variations.

As used herein, the terms “pore” and “pores” refer to pores of various sizes (also referred to as pore diameter or average pore diameter), including so-called “macropores” (pores greater than 50 nm diameter), “mesopores” (pores having diameter between 2 nm and 50 nm), “micropores” (pores having diameter of less than 2 nm) unless otherwise indicated, and “nanopores” (generally overlapping with microporous, mesoporous, and macroporous categories typically having pores with diameters between 2 nm and 100 nm), where the pore size refers to an average or median value. In certain aspects, the pores may be of a nanoscale dimension. A structure, like a pore, that is “nano-sized” or “nanometer-sized” may have at least one spatial dimension that is less than about 1 micrometer (i.e., 1,000 nm), optionally less than about 0.75 micrometers (i.e., 750 nm), optionally less than about 0.5 micrometers (i.e., 500 nm), optionally less than about 0.4 micrometers (i.e., 400 nm), optionally less than about 0.3 micrometers (i.e., 300 nm), optionally less than about 0.2 micrometers (i.e., 200 nm), and in certain variations, optionally less than about 0.1 micrometers (i.e., 100 nm). Accordingly, a nanopore may have at least one spatial dimension that is greater than about 1 nm and less than about 1,000 nm (1 micrometer). It should be noted that so long as at least one dimension of the nanopore falls within the above-described nano-sized scale (for example, diameter or a cross-sectional width), one or more other axes may well exceed the nano-size (for example, length).

In certain aspects, a plurality of pores may have an average pore size (also referred to as pore size, pore diameter or average pore diameter, corresponding to a major cross-sectional dimension, like pore diameter or width) of less than or equal to about 200 nm, for example, from greater than or equal to about 1 nm to less than or equal to about 150 nm, optionally greater than or equal to about 5 nm to less than or equal to about 100 nm, optionally greater than or equal to about 10 nm to less than or equal to about 80 nm, optionally greater than or equal to about 10 nm to less than or equal to about 70 nm, and in certain aspects, optionally greater than or equal to about 20 nm to less than or equal to about 50 nm.

Each of the pores may extend from a first surface of the membrane to a second opposite surface of the membrane to define a height that generally may correspond to a thickness of the membrane. As will be appreciated, the pore may extend through the body of the membrane such that it defines a channel extending from the first surface on the first side to the second opposite surface on the second side of the membrane and thus defines a height (h). In certain variations, both the height, as well as width and length, of the nanopores/nanochannels may be on a nanoscale.

In certain aspects, the porous insulating membrane may be selected to have a given geometry. It should be noted that porous insulating membrane defines an active region which has the desired geometry. In certain aspects, the active region is defined by the one or more regions of the membrane that are in contact with the electroporation fluid containing at least one cargo material and in contact with the cells that will receive the at least one cargo material. By way of example, the porous insulating membrane may itself define a first shape but it may be disposed adjacent to an opening of a receptacle for the electroporation fluid that has a distinct shape. Thus, the shape of the opening of the receptacle containing the electroporation fluid that contacts the porous insulating membrane may in fact be what defines the active region shape discussed herein. As will be appreciated, while the ensuing discussion refers to the shape of the porous insulating membrane, which in some variations may be defined by the membrane itself, it will be appreciated that in other variations, the shape of the membrane may in fact refer to a shape of an active region defined on the porous insulating membrane (e.g., determined by a shape of the opening on the receptacle containing electroporation fluid comprising the cargo material(s)). In various aspects, the active region of the membrane may have a cross-sectional shape with a perimeter-to-area ratio of greater than or equal to about 0.75.

In other aspects, the porous insulating membrane may be patterned, for example, with electrically non-conductive materials, like polymers, such as siloxanes like polydimethylsiloxane (PDMS) or an epoxy-based material, like a commercially available SU-8™ negative tone high contrast epoxy-based photoresist sold by MicroChem, which can generate geometric and/or edge effects in the delivery of the electric field across the membrane to the cells. In certain aspects, the pattern defines at least one continuous or discontinuous shape selected from the group consisting of: a polygon, a triangle, a rectangle, a square, a quadrilateral, a pentagon, a hexagon, a serpentine shape, and combinations thereof. These shapes may create an edge effect in an active region of the surface to enhance transport of the at least one cargo material via nanoporation into the cell. In certain aspects, the pattern may involve rectilinear patterns, which may include discontinuous rectilinear patterns, such as dotted, dashed, or discontinuous bars, pillars, or lines that define various rectilinear shapes or polygons. Alternatively, the pattern many include dotted arrays, hollow or solid features, and the like. For example, the rectilinear pattern may define a continuous or discontinuous polygon, triangle, rectangle, square, quadrilateral, pentagon, hexagon, serpentine shape, and equivalents and combinations thereof. The pattern optionally defines an array comprising a plurality of shapes repeated across the surface of the membrane at spaced intervals from one another. For example, the array may comprise a plurality of shapes selected from the group consisting of: a dot, a circle, a polygon, a triangle, a rectangle, a square, a quadrilateral, a pentagon, a hexagon, a serpentine shape, and combinations thereof. In one variation, the rectilinear pattern may comprise at least one discontinuous lines or dots However, such patterns may be any arbitrary pattern that facilitates edge effects across the membrane. The patterns may be formed by standard lithography techniques known in the art. In certain aspects, the patterns may be discontinuous lines.

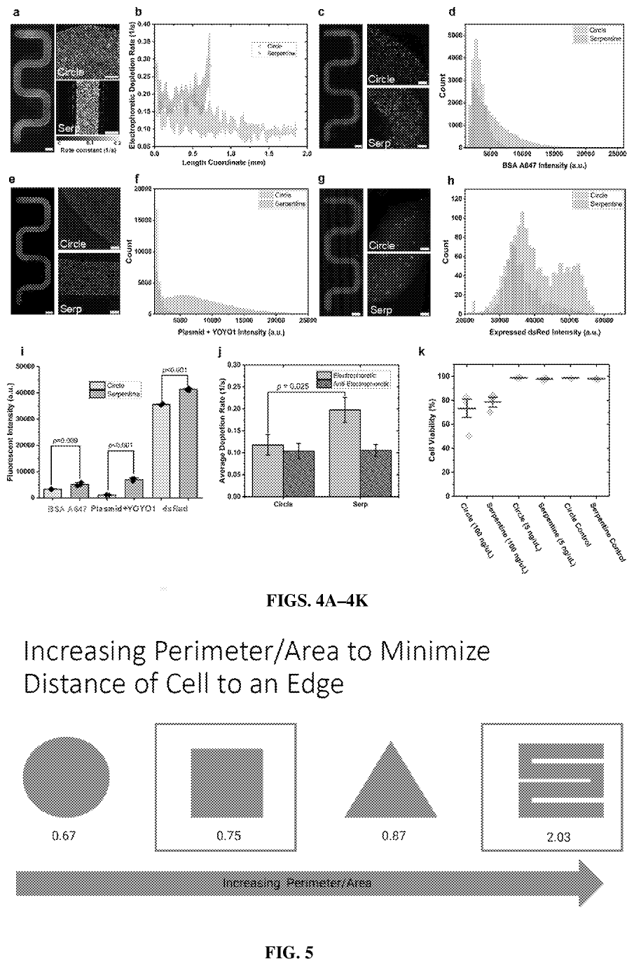

In accordance with certain aspects of the present disclosure and as shown in FIG. 5, the active region of the porous insulating membrane may define a cross-sectional shape for an active region that has a perimeter-to-area ratio of greater than or equal to about 0.75, for example, the perimeter-to-area ratio of a square, optionally greater than or equal to about 0.87, for example, the perimeter-to-area ratio of a triangle, optionally greater than or equal to about 1, optionally greater than or equal to about 1.5, and optionally greater than or equal to about 2, for example, the perimeter-to-area ratio of a square of a serpentine pattern shown in FIG. 5. Notably, a circle has a perimeter-to-area ratio of about 0.67. Thus, the shape (e.g., cross-sectional shape) of the active region of the membrane provided in the device may define a non-round shape or non-circular cross-sectional shape. For example, the membrane cross-sectional shape may have at least two sides or edges and optionally at least three sizes or edges, such as a polygon, like a rectangle, square, triangle, and the like. The cross-sectional shape may define one or more intersections between sides that define an angle, such as less than or equal to 90°, optionally less than or equal to about 75°, optionally less than or equal to about 60°, and in certain aspects, optionally less than or equal to about 45°. In other aspects, the angle may be obtuse, for example, having an angle of greater than or equal to about 90°, optionally greater than or equal to about 100°, optionally greater than or equal to about 110°, and in certain variations, optionally greater than or equal to about 120°. In certain aspects, a cross-sectional shape of the active region of the membrane may be selected from a group consisting of: polygonal, triangular, rectangular, squares, quadrilaterals, pentagons, hexagons, octagons, obtuse shapes, like a fan-shape or circular sector, irregular shapes, and combinations thereof. Notably, the cross-sectional shape may define a “serpentine” shape, meaning it has shape with at least two 90° changes in direction, optionally with at least two 180° changes in direction, which may include a spiral structure or an interdigitated type structure, by way of non-limiting example. The serpentine shape may have a perimeter-to-area ratio of greater than or equal to about 2. In certain aspects, serpentine may be considered to be a concave shape.

As will be discussed further below, the insulating porous membrane may be formed of a biocompatible material on which cells may be disposed and/or grown. The cells may be prokayrotic cells and/or eukaryotic cells. By way of non-limiting example, the cells may be bacterial, animal (including mammalian), plant, fungi, yeast, and the like. In certain variations, the cell may be a eukaryotic cell, such as an animal cell. The cells may be cells for various organs of a human or other mammal/animal, such as endothelial cells, cardiac cells, pulmonary cells, neural cells, connective tissue cells, vascular cells, stem cells, immune cells, cancer cells, and the like.

By “biocompatible,” it is meant that a material or combination of materials can be contacted with cells or tissue, in vitro or in vivo, or used with mammals or other organisms and has acceptable toxicological properties for contact and/or beneficial use with such cells, tissue, and/or animals. It will be recognized that “biocompatibility” is a relative term, and some degree of inflammatory and/or immune response is to be expected even for materials that are highly compatible with cells or living tissue. However, non-biocompatible materials are typically those materials that are highly toxic, inflammatory and/or are acutely rejected by the immune system, for example, a non-biocompatible material implanted into a subject may provoke an immune response in the subject that is severe enough such that the rejection of the material by the immune system cannot be adequately controlled, in some cases even with the use of immunosuppressant drugs, and often can be of a degree such that the material must be removed from the subject. In certain aspects, biocompatible materials are those that are approved for use in humans by an appropriate regulatory agency, such as the Federal Drug Administration (FDA) in the United States; the European Commission (EC)/European Medicines Agency (EMEA) in Europe; or Health Products and Food Branch (HPFB) in Canada.

In certain variations, the biocompatible material is a polymer. Suitable polymers include polycarbonate. For example, the membrane may be a nanoporous polycarbonate track-etched (PCTE) membrane.

The cell manipulation system also comprises at least one fluid or medium, considered to be an electroporation fluid composition. In certain aspects, the at least one cargo material to be delivered to at least a portion of the cells disposed on the porous membrane during the electroporation transfer process may be combined with the fluid or medium. The fluid medium or electroporation may be a liquid at the temperatures that the electroporation process is conducted. The fluid medium may include not only one or more cargo materials to be transferred to the cells, but also a carrier, such as an aqueous liquid comprising water or other polar solvents. Various electroporation mediums are contemplated. In one non-limiting example, the electroporation medium or buffer is commercially available from Bio-Rad as Gene Pulser Electroporation Buffer. However, in other aspects described further below, the electroporation fluid composition may be prepared in accordance with certain aspects of the present disclosure. For example, the electroporation fluid composition comprises a redox active compound, at least one cargo material to be delivered to or removed from at least one cell when a potential is applied to the electroporation fluid, and a carrier liquid. In certain variations, the fluid medium/electroporation fluid composition may be received in a receptacle having at least one opening that permits contact with the porous insulating membrane and may define the active region through which the at least one cargo material is delivered to at least one cell.

In certain variations, the present disclosure contemplates use of a new electroporation fluid composition. Such an electroporation fluid composition may be used for electroporation process of at least one cell, including a nanochannel or nanopore electroporation process, as described further here. The electroporation fluid composition may also be referred to as an electroporation buffer solution. The electroporation fluid may comprise a carrier liquid, which may be an aqueous liquid comprising water or other polar solvents. The electroporation fluid composition also comprises at least one redox active compound, as well as at least one cargo material to be delivered to the at least one cell when a potential is applied to the electroporation fluid, and a carrier liquid.

The redox active compound may be a conductive compound that further provides redox activity in the solution during the electroporation process. In certain variations, the redox active compound may be selected from the group consisting of: potassium ferrocyanide, potassium ferricyanide, ferrocenemethanol, and combinations thereof. In certain aspects, the redox active compound(s) may comprise ferrocyanide and/or ferricyanide. A cumulative concentration of the redox active compounds may be greater than or equal to about 1 mM to less than or equal to about 70 mM of the one or more redox active compounds, optionally greater than or equal to about 1 mM to less than or equal to about 50 mM of the one or more redox active compounds, optionally greater than or equal to about 1 mM to less than or equal to about 40 mM of the one or more redox active compounds, optionally greater than or equal to about 1 mM to less than or equal to about 30 mM of the one or more redox active compounds, optionally greater than or equal to about 1 mM to less than or equal to about 20 mM of the one or more redox active compounds, and optionally greater than or equal to about 1 mM to less than or equal to about 10 mM of the one or more redox active compounds, and alternatively optionally greater than or equal to about 2 mM to less than or equal to about 20 mM of the one or more redox active compounds, optionally greater than or equal to about 2 mM to less than or equal to about 10 mM of the one or more redox active compounds. In one non-limiting example, the electroporation fluid composition may have greater than or equal to about 1 mM to less than or equal to about 10 mM of potassium ferrocyanide and greater than or equal to about 1 mM to less than or equal to about 10 mM of potassium ferricyanide included as the redox active component. An additional redox active component that can supplement or complement the ferrocyanide is ferrocenemethanol, which may be present in the electroporation fluid composition at greater than or equal to about 1 mM to less than or equal to about 50 mM.

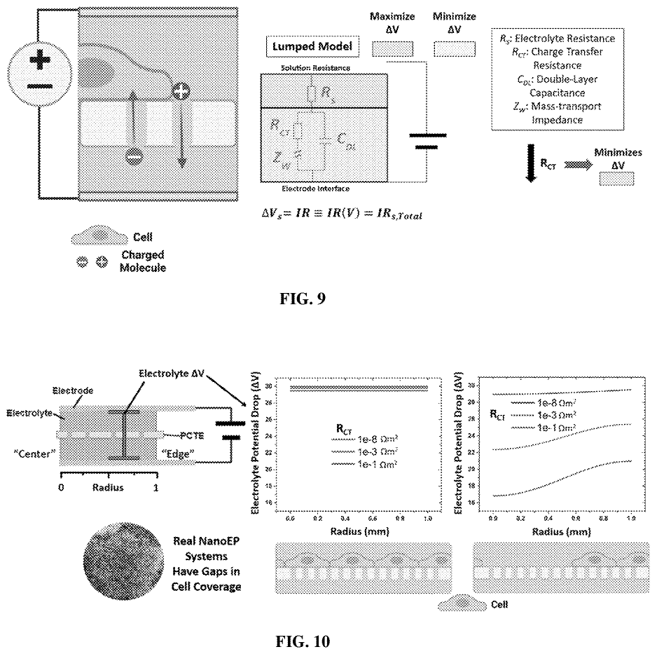

In various aspects, the electroporation fluid can be used in a cell manipulation system or device, for example, an electroporation or nanopore/nanochannel electroporation device like that shown in FIG. 9. The system includes a first electrode having a first polarity and a second electrode having a second opposite polarity. The first electrode and the second electrode are each formed of an electrically conductive material and are arranged in a spaced-apart relationship from one another, and may be formed in the same manner and of the same materials as described previously above.

The cell manipulation device may further include a porous membrane disposed between the first electrode and the second electrode, like that described above. The insulating porous membrane is configured to support and promote growth of cells (one cell is shown in FIG. 9). Each of the pores may extend from a first surface of the membrane to a second opposite surface of the membrane to define a height that generally may correspond to a thickness of the membrane. The cell manipulation system also comprises at least one fluid or medium, namely the electroporation fluid composition. The electroporation fluid may comprise a base of a conventional electroporation buffer or medium, such as Gene Pulser Electroporation Buffer, commercially available from Bio-Rad, in one non-limiting example, to which the redox active compound and the at least one cargo material may be added. The base fluid may have additional ingredients introduced, as well. In certain aspects, the at least one redox active material and the at least one cargo material to be delivered to at least a portion of the cells (e.g., disposed on the porous membrane) during the electroporation transfer process may be combined with the fluid or medium.

The electroporation medium/fluid composition may be held in a container or compartment having at least one opening where it is in contact with the cells in regions of the cell membrane that open so that the cargo can be transferred therein when an electric field or potential is applied to the first electrode and second electrode during the nanoporation or nanopore electroporation process. As will be described below, in certain aspects, a container or housing may be formed of a polydimethylsiloxane (PDMS) or other electrically insulating material that retains the at least one cargo material and/or fluid medium, for example, between the first electrode and the second electrode. The container or housing may have a cross-sectional shape that matches that of the membrane, so that the membrane may be disposed therein. In other variations, the shape of the opening of the container/receptacle containing the electroporation fluid that contacts the porous insulating membrane may define the active region shape on the membrane and may have a distinct shape from that of the perimeter of the membrane.

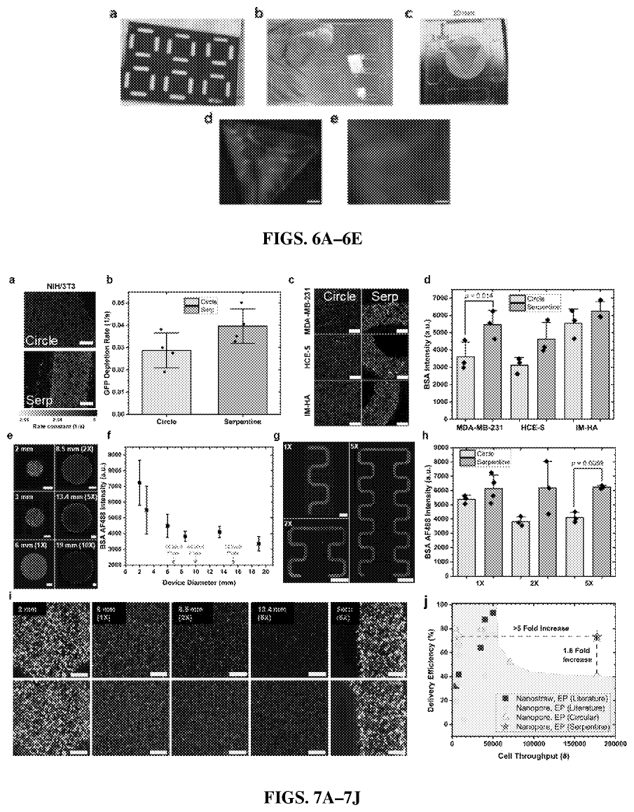

In certain aspects, to further enhance uniform cargo distribution beneath the nanoporous membrane during nanoporation/cargo delivery, pillar structures can be used to elevate the membrane from the adjacent (e.g., bottom) electrode. For example, FIGS. 6A-6C show a plurality of open corner squares each defined by four disconnected pillars, which may be about 200 micrometers tall and formed of a non-conductive material, such as PDMS. These pillars can be formed and cured and/or further treated (e.g., with plasma treatment for bonding) to couple them to the membrane elevate the membrane from the bottom electrode.

In various aspects, the redox-active compound in the electroporation fluid composition can help minimize or prevent electrode reactions with products that harm cells. Further, certain redox compounds can react with heavy metal ions to effectively chelate them. While performing either bulk electroporation (EP) or nanopore electroporation (NanoEP) as a cell transfection technique, electrodes will facilitate chemical reactions that negatively affect cell health or hinder cargo manipulation. These reactions cause pH changes, free radical generation, electrode ion leaching, and/or gas bubble formation. This unfavorable electrochemical environment can trigger cell death. Adding redox-active compounds in accordance with certain aspects of the present disclosure, for example, compounds comprising ferrocyanide and/or ferricyanide, such as potassium ferrocyanide and potassium ferricyanide (the combination of both compounds referred to herein as FeCN), can address all of the aforementioned issues. FeCN reduction/oxidation is an electrically driven reaction with very fast, reversible kinetics. It is easier for many electrode materials to catalyze the FeCN reactions over water splitting reactions, which prevent pH changes, free radical formation, and gas bubble formation. Further, FeCN dissolved in solution has a strong negative charge (e.g., −3/−4) and can react with heavy metal ions to prevent the ions from negatively affecting the cells.

Further, the redox-active compounds in the electroporation buffer fluid can increase the proportion of the applied electric field over the cells, which increases electromotive flux. Electroporation can be broken into an equivalent electrical circuit (NanoEP) shown in FIG. 9 with the cells lumped with the “solution resistance.” One objective is to maximize a voltage drop in the solution to maximize the electromotive force to deliver cargo molecules to cells. By lowering the electrode surface resistance (e.g., charge transfer resistance), with potassium ferro/ferricyanide as a mediator, consistently high flux from cells can be maintained, for example, greater than or equal to about 60% cargo removal or delivery efficiency), with greater than or equal to about 90% viability. This high flux is comparable to results obtained with commercial electroporation buffer (EP buffer) as a control, except the FeCN buffer is twice as conductive. With a more conductive buffer, the electromotive driving force over the solution is decreased, and thus a lower flux into cells would be expected. If the electroporation fluid/buffer is made more conductive with a non-redox active compound (potassium chloride), the flux decreases as expected. These unexpected results indicate the redox active component is effective at increasing electromotive flux despite being conductive.

The cargo material or payload to be transferred via transfection into the one or more cells may comprise a biomaterial, such as a biomolecule, or a gene therapy material or a cell therapy material. For example, a biomaterial may include a biofunctional agent that alters (e.g., promotes) cell growth, regeneration, differentiation, proliferation, and/or repair. By “promoting” cell growth, cell proliferation, cell differentiation, cell repair, or cell regeneration, it is meant that a detectable increase occurs in either a rate or a measurable outcome of such processes occurs in the presence of the biofunctional agent as compared to a cell or organism's process in the absence of such a biofunctional agent, for example, conducting such processes naturally. By way of example, as appreciated by those of skill in the art, promoting cell growth in the presence of a biofunctional agent may increase a growth rate of target cells or increase a total cell count of the target cells, when compared to cell growth or cell count of the target cells in the absence of such a biofunctional agent.

In certain variations, the biofunctional agent promotes cell growth, cell adhesion, cell proliferation, cell differentiation, cell repair, and/or cell regeneration by increasing a measurable process result (e.g., measuring total cell counts for cell generation or cell regeneration, measuring the rates or qualitative outcome of cell proliferation, cell differentiation, or cell repair rates) by greater than or equal to about 15% as compared to the result of the process in the absence of the biofunctional agent, optionally increasing by greater than or equal to about 20%, optionally increasing by greater than or equal to about 25%, optionally increasing by greater than or equal to about 30%, optionally increasing by greater than or equal to about 35%, optionally increasing by greater than or equal to about 40%, optionally increasing by greater than or equal to about 45%, optionally increasing by greater than or equal to about 50%, optionally increasing by greater than or equal to about 55%, optionally increasing by greater than or equal to about 60%, optionally increasing by greater than or equal to about 65%, optionally increasing by greater than or equal to about 70%, optionally increasing by greater than or equal to about 75%, optionally increasing by greater than or equal to about 80%, optionally increasing by greater than or equal to about 85%, optionally increasing by greater than or equal to about 90%, and in certain aspects, optionally increasing by greater than or equal to about 95%.

In certain aspects, the biomaterial may be selected from the group consisting of: a pharmaceutical active ingredient, a protein, a peptide, a growth factor, a biofactor, a peptide, a cytokine, a hormone, a genetic payload, such as a gene therapy material, a cell therapy material, and combinations thereof. Biofunctional active ingredients or agents may also include nanomaterials and/or imaging agents, by way of non-limiting example.

Suitable cell therapy materials may be a biomaterial, which may in certain aspects promote cellular growth or regeneration, which can affect or promote cell recruitment, migration, attachment, proliferation, differentiation, maturation, apoptosis, and the like. Suitable gene therapy materials include a genetic engineering agent that may be delivered to a cell and can modify one or more genes or the expression of one or more genes in at least one cell growing or supported on or within the tissue scaffold. For example, the gene therapy material may add, remove, or modify nucleic acids (e.g., DNA) of genes to result in a genetic modification to the cells. The gene therapy material may comprise a polynucleotide agent that when transferred and delivered to the cell achieves genetic modification or manipulation of the cell. By way of example, polynucleotide agents may be selected from the group consisting of: DNA, RNA, plasmids, plasmid DNA (pDNA), virus vectors, short interfering sequence of double stranded RNA (siRNA), messenger RNA (mRNA), transfer RNA, ribosomal RNA, small nuclear RNA, single stranded DNA, microDNA, guide RNA, CRISPR CAS-9, aptamers, and combinations thereof. In certain variations, the cell DNA may be modified, for example, by using CRISPR CAS-9, whereas in other cases, a polynucleotide agent, like plasmid DNA (pDNA), can encode for generating a protein.

Additionally, the electroporation fluid composition may comprise one or more conventional electroporation components, such as, for example, a salt, a sugar, a buffering agent, a chelating agent, equivalents and combinations thereof. By way of non-limiting example, a sugar, such as a monosaccharide or disaccharide, like sucrose, glucose, trehalose, mannitol, and any combinations thereof may be included. Salts may include various salts used in electroporation buffer compositions, such as a halide salt of an alkali or alkaline earth metal, like chlorine salts, including potassium chloride (KCl) and/or magnesium chloride (MgCl₂). One or more buffering agents may be included, such as 4-(2-hydroxyethyl)-1-piperazineethanesulfonic acid (HEPES), dimethyl sulfoxide (DMSO), dibasic sodium phosphate (Na₂HPO₄), monobasic sodium phosphate (NaH₂PO₄), or combinations thereof. Chelating agents may form a chelating complex with an alkali or alkaline earth metal cation from the halide salt. Examples of suitable chelating agents are ethylenediamine tetraacetic acid (EDTA) and/or ethylene glycol tetraacetic acid (EGTA). Notably, one or more pH adjusting compounds or buffers may be introduced to adjust pH to a desired range, such as potassium phosphate (e.g., monopotassium phosphate (KH₂PO₄), dipotassium phosphate (K₂HPO₄), tripotassium phosphate (K₃PO₄)), or potassium hydroxide (KOH), hydrochloric acid (HCl), and the like. Other optional components may include adenosine triphosphate (ATP), by way of non-limiting example. As noted above, in certain embodiments, the carrier or solvent of the electroporation fluid composition comprises water.

In one variation, the base composition of the electroporation fluid composition comprises greater than or equal to about 5 mM to less than or equal to about 15 mM potassium phosphate buffer, greater than or equal to about 10 mM to less than or equal to about 20 mM of 4-(2-Hydroxyethyl) piperazine-1-ethane-sulfonic acid (HEPES) buffer. Further, the electroporation fluid composition may have greater than or equal to about 125 mM to less than or equal to about 200 mM of sugar (e.g., sucrose, trehalose, etc.). Finally, the electroporation fluid composition may have greater than or equal to about 1 mM to less than or equal to about 10 mM of potassium ferrocyanide and greater than or equal to about 1 mM to less than or equal to about 10 mM of potassium ferricyanide included as the redox active component. An additional redox active component that can supplement or complement the ferrocyanide is greater than or equal to about 1 mM to less than or equal to about 50 mM ferrocenemethanol. Optional additional components for the electroporation fluid composition include greater than or equal to about 1 mM to less than or equal to about 5 mM of adenosine triphosphate (ATP) and greater than or equal to about 0.1 vol. % to less than or equal to about 1 vol. % of dimethyl sulfoxide (DMSO). A pH range of the electroporation fluid composition may be greater than or equal to about 7 to less than or equal to about 7.5, with pH adjustments using potassium hydroxide or hydrochloric acid as needed. An electrical conductivity of the electroporation fluid composition may be greater than or equal to about 0.1 to less than or equal to about 0.5 S/m.

Thus, in certain variations, the present application contemplates a method of conducting electroporation (EP) or nanopore electroporation (NanoEP) as a cell transfection technique to either deliver or remove a cargo material from one or more cells. For example, the NanoEP uses a nanoporous membrane such as a polycarbonate track-etched (PCTE) membrane, to localize an electric field to temporarily form pores throughout the cellular membranes of cells that are in contact with the nanopores of the PCTE membrane in an electroporation cell manipulation device. This NanoEP transfection method is highly efficient and safer to cells compared to bulk electroporation. The cells may be eukaryotic cells, for example, animal cells like mammalian cells. In certain aspects of the present disclosure, the use of an electroporation fluid comprising a redox-active compound enhances the delivery efficiencies and/or depletion rates in an electroporation/nanopore electroporation process, while enhancing cell survival after the transfection/electroporation process. In certain aspects, a high flux to or from cells can be maintained, for example, greater than or equal to about 60% cargo removal or delivery efficiency, optionally greater than or equal to about 65%, optionally greater than or equal to about 70%, optionally greater than or equal to about 75%, optionally greater than or equal to about 80%, optionally greater than or equal to about 85%, and in certain aspects, optionally greater than or equal to about 90%. Further, cell survival after conducting the electroporation or nanopore electroporation transfection in the presence of an electroporation fluid prepared in accordance with certain aspects of the present disclosure, for example, having a redox-active compound, may be greater than or equal to about 70% cell survival, optionally greater than or equal to about 75%, optionally greater than or equal to about 80%, optionally greater than or equal to about 85%, optionally greater than or equal to about 90%, optionally greater than or equal to about 95%, and in certain aspects, optionally greater than or equal to about 97% cell survival.

As further background, intracellular molecular delivery of nucleic acids, or transfection, is essential for biomedical applications such as gene therapy, tissue engineering, and disease modeling. While various transfection techniques exist, there are trade-offs between efficiency, throughput, and dosage control when selecting an appropriate method. In vitro and ex situ transfection methods can be broadly categorized as substrate-based or substrate-free. While substrate-free methods are generally considered high throughput, substrate-based transfection techniques have gained attention due to their ability to enhance intracellular delivery by leveraging engineered cell-material interfaces. However, despite their advantages, substrate methods still face challenges in achieving uniform and predictable transfection across a cell population.

However, the present application contemplates enhancing the transfection ability and cell viability as discussed above. In certain variations, a technique to pattern cell monolayers using nanopore electroporation (NanoEP) as a cell transfection technique, alongside a patterning substrate as a membrane. The NanoEP uses a nanoporous membrane such as a polycarbonate track-etched (PCTE) membrane, to localize an electric field to temporarily form pores throughout the cellular membranes of cells that are in contact with the nanopores of the PCTE membrane. This transfection method is highly efficient and safer to cells compared to bulk electroporation. In certain aspects of the present disclosure, the polycarbonate track-etched membrane has different geometries of pores, which can create gradients of gene expression. In certain variations, the geometry of the active region of the device/membrane (e.g., square, triangle, circle) can drive delivery and depletion of cargo/biomolecules from the medium as a function of shape. The presence of edges in the active region of the membrane, more specifically a distance of a cell to an edge, enhances the delivery efficiencies and depletion rates in nanopore electroporation, which can be used to create complex cell monolayers. The present disclosure contemplates both non-uniform delivery based on geometric properties of the shape (for example using low perimeter to surface area ratio based on the shape of the device) with high level of fidelity. Similarly, membrane geometry has been leveraged for highly controlled delivery/depletion (e.g., serpentine channels, which have high perimeter to surface area ratio). Lastly, methods of integrating patterns to generate “local defects” on the membrane are contemplated by simply incorporating distributed patterns of a low electrical conductivity material, such as polydimethylsiloxane (PDMS) or other low conductivity polymers, such as dots, or thin grids to control the macromolecular delivery/depletion over large surface areas with uniform delivery for highly scalable delivery (transfection) and depletion (sampling) techniques.

Thus, the present disclosure contemplates utilizing different geometries (e.g. square, triangle, circle) patterned on PCTE membranes for nanopore electroporation to create gene expression gradients on cell monolayers. In certain aspects, the membrane itself may have a non-round/non-cylindrical channel cross-sectional shape. For example, the shape may define a cross-sectional shape that has at least two sides, such as a polygon, like a rectangle, square, triangle, and the like. In certain other aspects, the cross-sectional shape may be round, defining a circle, oval, or the like, although other irregular shapes are also contemplated. In such variations, while the membrane shape may be round or oval, the surface may be patterned to control geometry or the active region may define a shape defining a cross-sectional shape having a perimeter-to-area ratio of greater than or equal to about 0.75. In certain aspects, a cross-sectional shape of the membrane/active membrane be selected from a group consisting of: oval, spherical, round, polygonal, triangular, rectangular, squares, quadrilaterals, pentagons, hexagons, irregular shapes, and combinations thereof. An active region of the membrane may have a cross-sectional shape selected from a group consisting of: a polygonal, triangular, rectangular, squares, quadrilaterals, pentagons, hexagons, irregular shapes, and combinations thereof, by way of example. As described herein, the present technology provides efficient biomolecule delivery, driven by and dependent on geometry, measured through depletion rates and biomolecule uptake into cells. The delivery efficiency and depletion rates are enhanced with the presence of more edges, specifically cell distance to edges of the pore in the membrane, enabling creation of complex cell monolayers of cells. Further, non-uniform and highly controlled delivery is provided, based on low and high perimeter to surface area ratios, respectively, of biomolecule delivery to cells is available. Additionally, given the capacity of this technology to leverage geometry for highly controlled uniform biomolecule delivery, it is highly scalable for transfections and sampling techniques over large surface areas. As described above, “local defects” can also be intentionally designed through incorporation of distributed patterns on the membrane, for example, by using PDMS or other low conductivity polymers to form such patterns.

Thus, the present disclosure contemplates improved uniform biomolecule/cargo material delivery over current transfection methods to cell monolayers, leveraging geometrical membrane shape for optimization. Further, different cell types may readily uptake biomolecules based on membrane geometry (over current transfection methods). Additionally, as noted above, “local defects” introduced on the membrane surface can provide intentional non-uniform delivery of biomolecules-within a cell monolayer that includes other areas of highly controlled efficient biomolecule delivery. For example, regions may be coated with insulating patterns to enhance localized electric fields. The present technology is high scalable, including in industrial settings where various biomolecules—including DNA, RNA, proteins, peptides, and even nanomaterials—can be used on cell monolayers.

As further background, intracellular molecular delivery of nucleic acids, or transfection, is essential for biomedical applications such as gene therapy, tissue engineering, and disease modeling. While various transfection techniques exist, there are trade-offs between efficiency, throughput, and dosage control when selecting an appropriate method. In vitro and ex situ transfection methods can be broadly categorized as substrate-based or substrate-free. While substrate-free methods are generally considered high throughput, substrate-based transfection techniques have gained attention due to their ability to enhance intracellular delivery by leveraging engineered cell-material interfaces. However, despite their advantages, substrate methods still face challenges in achieving uniform and predictable transfection across a cell population.

As described above, nanopore electroporation (NanoEP) is a substrate-based transfection method, which employs an insulating substrate (in the horizontal x-y plane) with vertically aligned nano-channels to localize the electric field (applied vertically along the z-axis) at the cell-nanochannel interface for cells that are disposed thereon. This field localization along the z-axis forms temporary electropores in the cell membrane and allows for precise electrophoretic transport of cargo material into and out of cells, which makes NanoEP a highly efficient non-viral transfection method. Bulk electroporation—NanoEP's substrate-free analogue—exposes suspended cells uniformly to high electric fields (>500 V/mm) and forms electropores along the entire cell membrane, causing extensive cell damage and unwanted transcriptional outcomes. NanoEP overcomes these issues and achieves high delivery efficiency (e.g., greater than 60%) and high cell viability (e.g., greater than 90%) at lower voltages (<15 V/mm).

Like most substrate-based transfection, the throughput of NanoEP scales with the 2-dimensional (2D) area of the substrate the target cells interface with—at least in principle. While NanoEP has been primarily studied in the context of its vertical (z-axis) electric field effects, the impact of the lateral (x-y) electric field distribution on transfection uniformity remains largely unexplored. The efficiency and scalability of NanoEP are directly tied to the spatial uniformity of cargo delivery and depletion across a cell population. Some studies have shown that the geometry of electrodes affects the electric field distribution if the electrode has a small geometric surface area parallel to the growth substrate. Additionally, it has been previously demonstrated that cargo transport in NanoEP is primarily governed by electromigration, rather than diffusion. Thus, in accordance with certain aspects of the present disclosure, optimizing the lateral electric field distribution is believed to enable precise control over transfection uniformity, allowing substrate-based electroporation, when used as a vertical stack, to scale comparably to its 3D, substrate-free counterparts.

However, as discussed further herein, the lateral spatial uniformity of the z-focused electric field in NanoEP processes desired for scaled-up, ex vivo transfection applications cannot be simply assumed. An “edge effect” is identified in conjunction with the present teachings, where cells near the perimeter of the membrane experience different delivery efficiencies compared to those in the center, highlighting a key challenge in achieving uniform transfection across large-scale NanoEP systems. This phenomenon is believed to likely be due to non-uniform electric fields created by the geometry of the membrane itself. Understanding and controlling these lateral electric field effects can enable the creation of highly uniform or intentionally heterogeneous cell populations, important for applications such as tissue engineering, where spatially controlled gene expression is necessary for modeling complex biological systems.

In the following examples, the impact of membrane geometry on the lateral electric field distribution in NanoEP systems is investigated. The presence of edge effects is identified and characterized to develop a theoretical framework using the Nernst-Planck model/equation based on both experiments and a theoretical model to predict kinetic rates for cargo depletion and geometric trends in cargo delivery. The results demonstrate that by modifying membrane geometry, specifically by adjusting perimeter-area ratios and introducing angular variations, intracellular molecular delivery gradients can be tuned within cell populations. Further, these geometrically anisotropic electric fields can result in the over delivery of cargo in certain areas, which leads to cell death. Thus, the present disclosure contemplates leveraging membrane geometry as a design parameter in NanoEP systems to provide a scalable and tunable approach for creating complex 2D tissue models and improving uniformity in transfection-based applications.

As described further in the examples, a modified Nernst-Planck model is developed to guide design of membrane geometries that either promote delivery uniformity or create prescribed spatial gradients within cell monolayers. By varying the internal angles formed by the membrane edges (60°, 90°, 120°), predictable intracellular cargo gradients are created, while concave “serpentine” geometries with high perimeter-to-area ratios amplify delivery efficiency and minimize spatial heterogeneity. The present disclosure contemplates a serpentine NanoEP device design that achieves a significant improvement in cargo flux for scaled-up operation in multiple cell types using a diverse set of payloads, allowing up to an approximate 6-fold increase in an amount of cargo material delivered to be achieved and an approximate 2-fold increase in cargo material removal rates over the currently used geometry when scaling up cell engineering throughput. As explored further herein, large-area NanoEP systems suffer from a substrate dependent “edge effect,” in which cells near the perimeter of the nanoporous membrane exhibit enhanced delivery and faster cargo depletion than those at the center. This non-uniformity is attributed to a lateral electric field confinement effect created by the geometries of the cell-membrane in tandem with the electrolyte-electrode interfaces. Understanding and controlling these effects are essential for scaling NanoEP-based systems and could enable the creation of not only highly uniform but also intentionally heterogeneous cell populations, which is critical in tissue engineering where spatially controlled gene expression and protein loading is necessary for modeling complex biological systems.

As described in the examples, how device geometry influences lateral electric field distributions in NanoEP is explored. Using the Nernst-Planck equation, observed edge effects are quantified and design rules to predict kinetic rates for cargo depletion are identified and geometric trends in cargo delivery. The results demonstrate that by modifying membrane geometry, specifically by introducing angular variations in membrane edges and adjusting perimeter-to-area ratios, intracellular molecular delivery and depletion gradients can be tuned within 2D cell populations. These findings suggest that membrane geometry can serve as a tunable design parameter in NanoEP systems to enable both uniform and intentionally patterned intracellular payload delivery or depletion, offering a scalable strategy for next-generation, high-throughput transfection platforms and synthetic tissue constructs.

### Example 1

Various embodiments of inventive technology can be further understood by the specific examples contained herein. Specific Examples are provided for illustrative purposes of how to make and use the compositions, devices, and methods according to the present teachings and, unless explicitly stated otherwise, are not intended to be a representation that given embodiments of this invention have, or have not, been made or tested. Figures discussed herein were generated in bio render.

### Device Fabrication

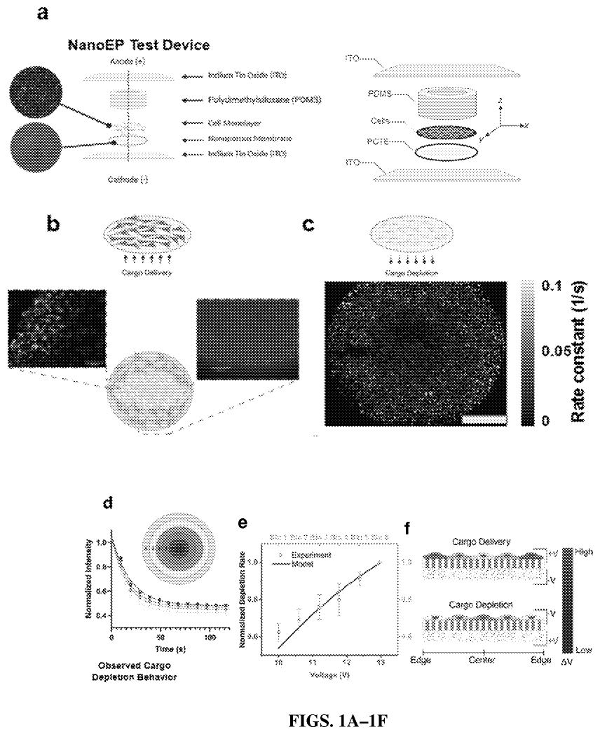

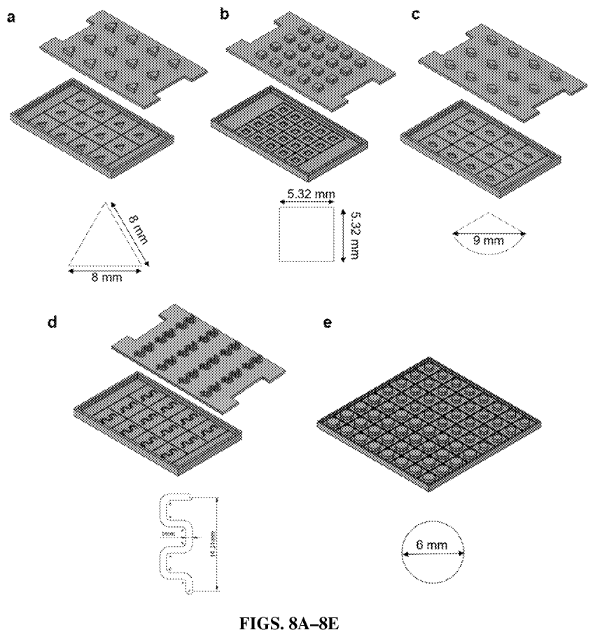

An example of a cell manipulation device like that shown in FIG. 1A is formed as follows. Molds of different geometries are designed with Fusion 360 and 3D printed with the Asiga Pro 4k45 DLP printer. See FIGS. 8A-8E showing the 3D models for each geometry where devices have a total thickness of 2 mm. FIG. 8A shows a two-layer click in mold design for triangle-shaped membranes for devices, where each side is 8 mm. FIG. 8B shows a two-layer click in mold design for square-shaped membrane devices, where each side is 5.32 mm. FIG. 8C shows a two-layer click in mold design for obtuse-shaped (e.g., circular sector shaped) devices, where a major dimension is 9 mm. FIG. 8D shows two-layer click in mold design for serpentine-shaped membrane devices, where a thickness of the serpentine shape is about 1 mm and an overall length of the serpentine channel is 14.31 mm. Finally, FIG. 8E shows a mold for conventional circular-shaped devices used in the examples for comparison, where a diameter is 6 mm. A two-layer click in mold design was not used for the circular geometry, because a 6 mm biopsy punch was instead used to clean up any devices that had thin films of cured PDMS on the circular region.

DentaMODEL (Asiga) resin is used for the printing material. Post-printing, the 3D printed molds were cured with UV light for 2 minutes, followed by a 30-minute isopropyl alcohol (IPA) wash, and then baked overnight at 60 °C. The area of the device geometries was kept constant to 28.3 mm² as well as a height of 2 mm unless specified otherwise. Polydimethylsiloxane (PDMS Sylgard™ 184 DOW) at a 10:1 ratio (base to curing agent) was cast into the molds and cured overnight at 60 °C. To attach the polycarbonate track etched membranes (PCTE) of 200 nm (calcein depletion, propidium iodide and BSA delivery) or 800 nm (plasmid delivery) pore diameters (Cytiva Whatman) to the PDMS devices, uncured PDMS was spun coat on a glass slide to create a 20 μm PDMS layer. Devices were then tapped onto the glass slide to coat the device surface with uncured PDMS, and the PCTE membranes were then placed on the device and cured overnight at 60 °C. For delivery experiments, pillars were added underneath the device using the same technique of tapping uncured PDMS to the pillars and curing overnight at 60 °C. Indium tin oxide (ITO) glass slides (Nanocs, 5 Ω/sq) were used as both the positive and negative electrodes.

### Cell Culture and Seeding in Devices

HT-1080 cells, a fibrosarcoma cell line (provided by the Thurber lab at the University of Michigan), were cultured in ATCC Eagle's Minimum Essential Medium (EMEM) with 10% fetal bovine serum (FBS) (Gibco) and 1% penicillin streptomycin (P/S) solution (Gibco). Human Corneal Epithelial Cells (HCE-S) were cultured in PriGrow III (TM003), 10% FBS, non-heat activated (Gibco), 1× GlutaMAX (Gibco), and 1% P/S solution (Gibco). Immortalized Human Astrocytes (IM-HA) were cultured in Astrocyte Medium, including 2% FBS, 1% Astrocyte Growth supplement, and 1% P/S solution (Innoprot). NIH/3T3 cells (cytosolic GFP expression), an embryonic mouse fibroblast cell line, were cultured in Dulbecco's Modified Eagle's Medium (DMEM) (Gibco) with 10% FBS and 1% P/S solution (Gibco). U2OS cells, a human osteosarcoma cell line, were cultured in ATCC McCoy's 5A Medium with 10% FBS and 1% P/S solution. Epithelial breast cancer cells (MDA-MB-231) were cultured in DMEM (Gibco) with 10% FBS and 1% P/S solution. On Day 0, the devices were treated with oxygen plasma for 1 minute (Plasma Etch, Inc.) for sterilization and increasing wettability. The devices were then coated with 27.5 μg/mL of fibronectin, from human plasma (Sigma-Aldrich), in phosphate buffered saline (Gibco) and incubated for 1 hour. Following fibronectin incubation, the devices were washed with media and HT-1080 cells were seeded in the devices at a cell density of 125,000 cells/cm². Cells were electroporated the following day.

### Electroporation: Cargo Delivery and Depletion

All cells were electroporated one day after cell seeding (Day 1) with Gene Pulser Electroporation Buffer (Bio-Rad). Cells were exposed to the buffer for roughly 10 minutes. A VSP-300 Potentiostat (BioLogic) was used for applying the electrical pulses. The bottom ITO electrode was the cathode (−) and the top electrode was the anode (+). The electroporation buffer or cargo solution was pipetted on the bottom electrode at a volume of about 10 to about 100 microliters (μL). The device was then placed on top of the droplet and overfilled with electroporation buffer to prevent any air gaps when placing the top electrode above the device. Copper tape was used to connect the alligator clips to the ITO slides.

For delivery of Bovine Serum Albumin (BSA) Alexa Fluor™ 488 and 647 conjugates (Invitrogen™), the cells were pre-stained with Hoechst 33342 (Invitrogen) at 15 micrograms (μg)/mL, and fluorescent BSA cargo solution was placed at a concentration of 2.5 mg/mL in the electroporation buffer. Before placing the device on the cargo solution, the device was dried with a Kimwipe® delicate task wipe available from Kimberly-Clark to ensure the cargo solution would not be diluted from the residual media on the device. The cells were electroporated at an applied voltage of 15-30 V at 20 Hz and 1 ms pulse square wave pulse widths for 5 to 20 seconds. BSA delivery images were taken around 15 minutes and approximately 1 hour after electroporation on the ECHO Revolve microscope or the Cytation 5 (Biotek Agilent). Delivery efficiency was calculated from dividing positive BSA-AF488 signal (positive BSA-AF488 signal corresponds to cell intensities being greater than 2000 a.u.) by total cell count (Hoechst). For depletion of calcein, cells were pre-stained with calcein AM (Invitrogen) at 3 micromoles (μM) for 30 minutes, approximately 1 hour before electroporation. The cells were electroporated at an applied voltage of 15 V at 20 Hz and 0.2-1 ms pulse widths for 60-120 s. For electrophoretically driven calcein removal, the top electrode was the cathode (−) and the bottom electrode was the anode (+), imaging through the nanoporous membrane to avoid imaging the cathode reduction. For anti-electrophoretically driven calcein removal, the top electrode was the anode (+) and the bottom electrode was the cathode (−), imaging above the nanoporous membrane to avoid imaging the cathode reduction. Calcein depletion experiments were recorded in real time on the ECHO Revolve fluorescent microscope.

For delivery of plasmid, pLenti3.7-DsRed plasmid (5 MDa) was stained with YOYO-1 dye (Biotium) at a ratio of 10 base pairs:1 YOYO-1 molecule. The solution was left to incubate at 37 °C for 2 hours. A PCR purification kit (GeneJET, Thermo Scientific) was used to separate unbound YOYO-1 molecules from the solution of bound YOYO-1 molecules to the plasmid. Solutions were diluted with an electroporation buffer at concentrations of 5 to 100 ng/μL. Before placing the device on the cargo solution, the device was dried with a kimwipe to ensure the cargo solution would not be diluted from the residual media on the device. The cells were electroporated at an applied voltage of 25 V, 1 Hz, 10 ms square-wave pulses for 4 s duration. Cell viability was taken 24-48 hours after electroporation with Cy5 NucSpot® Nuclear Stain (Biotium) and Hoechst 33342 (Invitrogen). It was then quantified using CellProfiler.

For delivery of plasmid, pLenti3.7-DsRed plasmid (5 MDa) was stained with YOYO-1 dye (Biotium) at a ratio of 10 base pairs:1 YOYO-1 molecule. The solution was left to incubate at 37 °C for 2 hours. A PCR purification kit (GeneJET, Thermo Scientific) was used to separate unbound YOYO-1 molecules from the solution of bound YOYO-1 molecules to the plasmid. Solutions were diluted with an electroporation buffer at concentrations of 5 to 100 ng/μL. Before placing the device on the cargo solution, the device was dried with a kimwipe to ensure the cargo solution would not be diluted from the residual media on the device. The cells were electroporated at an applied voltage of 25 V, 1 Hz, 10 ms square-wave pulses for 4 s duration. Cell viability was taken 24-48 hours after electroporation with Cy5 NucSpot® Nuclear Stain (Biotium) and Hoechst 33342 (Invitrogen). It was then quantified using CellProfiler.

For cargo delivery cell intensity profile cell analysis and delivery efficiency analysis, Biotek Agilent Gen5 software was used. Background subtraction was performed on the images and two different cell recognition techniques were used

### ITO Darkening Reaction Experiment

For imaging the darkening/reaction of the cathode, 2 mm and 6 mm diameter PDMS well devices (2 mm tall) were used with or without a membrane with an applied voltage of 25 V at 20 Hz frequency for 1 ms pulse widths for a total duration of 60 seconds. In other variations, an applied voltage of 30 V, 20 Hz, 1 ms square-wave pulses for 40-60 s duration is used. No cells were used, and Gene Pulser Electroporation Buffer was used as the buffer. Images were analyzed using MATLAB to measure the darkening over time.

### Potentiostat Data Collection

Biologic's EC Lab software was used to control the electrical parameters of the NanoEP experiments. Prior to running the electroporation experiment, potentio-electrical impedance spectroscopy (PEIS) was conducted over frequencies ranging from 2 MHz to 500 mHz, with a sine amplitude of 10 mV and base potential of 0 V DC. No reference electrodes were used in this study. PEIS was used to elucidate the solution/device resistance near 100-1 kHz, the x-coordinate where the imaginary (y-coordinate) Nyquist impedance is ~0Ω. PEIS was also conducted after the NanoEP experiment to see if the device resistance changed. NanoEP was conducted using the software's differential pulse amperometry (DPA) technique. Average currents reported are only when the pulse is applied, not an average of the on and off state.

### COMSOL Simulation and Circuit Modeling

The COMSOL geometry was created and evaluated using the Primary Current Distribution package. All simulations were run at 37 °C. The electrolyte was modeled as water, with a fixed conductivity of 0.2 S/m. Electrodes for the 3-D geometry were ITO and for all simulations, unless noted, were 100 nm thick, with a conductivity of 3×10⁶ S/m. The simulation incorporated a “surface resistance” node at the electrode/electrolyte interface (EEI; ave, with an experimentally measured value of 0.0014-0.0015 Ω·m². This value was similar for all tested device geometries. The surface resistance was experimentally determined by calculating the average voltage drop over the Electrode Electrolyte Interface (EEI; averaged among several devices of a certain shape), dividing by the current through the system, and multiplying it by the surface area. In all simulations, both electrodes were matched to have the same surface resistance and capacitance. In certain aspects, the bottom electrode is much closer to the polycarbonate membrane (~0.1 mm), to mimic experimental setups where a PDMS device is placed on a large droplet of electroporation buffer for cargo delivery/removal. The top electrode is separated from the bottom by approximately 2 mm. The polycarbonate is 15 μm in thickness and has a conductivity of 1× 10⁻¹⁷ S/m.

Equivalent circuit modeling was performed using the LTspice software. Built-in modules for circuit elements were used, and a square pulse element was used to apply the voltage. No product specifications were added to the circuit elements.

### SEM Imaging

SEM imaging was performed using the Thermo Fisher Nova 200 Nanolab at the Michigan Center for Materials Characterization (MC²). The ITO coated glass was mounted using conductive carbon tape and grounded using colloidal graphite glue (Electron Microscopy Sciences). Images of ITO surfaces were acquired using secondary electrons at 5 kV and 1.6 nA of beam current.

### Nernst Planck Simulation

To leverage simulated voltage profiles to easily predict cargo depletion rates, a simplified Nernst Planck (NP) equation was implemented in Python. The movement of charged molecules in solution is described by the NP equation, shown in Equation 1. An equivalent form of the NP equation can be found according to the divergence theorem, so that the number (nₚ) and area (Aₚ) of electropores can be considered in the analysis. The flux term (J) which incorporates the effects of concentration (c), fluid velocity (v), electric field (E), temperature (T), and cargo diffusivity in water at 37 °C (D_{AB}). The electric field is related to flux using the molecule charge (z), elemental charge (e), and Boltzmann constant (k_{b}).

$$J = - D ∇ c + cv + \frac{- Dze}{{k}_{b} T} c \frac{d ψ}{dz} \qquad ( 1 )$$

$$\frac{\partial c}{\partial t} + ∇ \cdot J = 0 ⇔ V \frac{dc}{dt} + {JA}_{p} {n}_{p} = 0 \qquad ( 2 )$$

$$J = - D ∇ c + cv + \frac{- Dze}{{k}_{b} T} c \frac{d ψ}{dz} \qquad ( 3 )$$

The flux of the molecules (J) is the vector sum of contributions from diffusion, advection, and electromigration. Eq. 3. The equation was simplified by removing the effect of advection (there is no fluid velocity) and calcein diffusion. Molecular diffusion was neglected since the calculated electromotive species flux across the nanoporous membrane was approximately 1,350 times greater than that of diffusion and dominates cargo transport. The flux is then plugged into a general mass balance in Eq. 2 with no assumed reaction and a transient concentration of cargo inside the cell volume (V). Here, Aₚ represents the area of one electropore on the cell and nₚ is the number of electropores. When the simplified Equation 3 is plugged into Equation 2 and integrated with respect to time, it yields Equation 4.

$$C ( t ) = {C}_{0} {e}^{( - [ \frac{{DzeA}_{p} {n}_{pd φ}}{{Vk}_{b} Tdz} ] t )} ≡ {C}_{0} {e}^{- ( \frac{Dezd φ1}{{k}_{b} {Tdzl}_{eff} ( φ )} ) t} ≡ {C}_{0} {e}^{- kt} \qquad ( 4 )$$

$${l}_{eff} ( φ ) = \frac{V}{{A}_{p} ( φ ) {n}_{p} ( φ )} [ = ] m \qquad ( 5 )$$

The electropore area, number of electropores, and the cell volume are lumped into a fitting parameter l_{eff}, as these values can be difficult to ascertain or are uncertain during the experiment. This yields an interpretable meaning for the experimental calcein rate contents. This parameter is used to adjust the timescale of the simulation equation to match experimental data by fitting l_{eff} to minimize the mean absolute error. This fitting was only done once to develop a linear mapping from voltage input space to the kinetic rate output space. This mapping was not adjusted between devices to demonstrate the predictive power of this simplified model on several real systems.

This lumped model for charged molecule transport is mapped onto device geometry by utilizing the electric field at each point in space retrieved from COMSOL simulations in a 3-D NanoEP device. The NP simulation is 0-D and treats the hypothetical cell as a point in space, but input voltages from different (x, y) points are used in the COMSOL solution space to effectively predict calcein depletion rates over the entire membrane geometric area.

Evaluation of Cargo Depletion and Decay in Rate Constant/Intensity Distance from Edge/Corner

Cargo depletion was assessed by tracking the reduction in fluorescent signal intensity of cells pre-stained with calcein AM during time-lapse imaging, conducted using the ECHO Revolve microscope. The fluorescent intensity drop is modeled by exponential decay shown in Equation 6 below:

$${I}_{f} ( t ) = ( {I}_{f , 0} - b ) {e}^{- kt} + b \qquad ( 6 )$$

I_{f} is the fluorescence intensity of each pixel (or bin) tracked over time (t), starting at an initial fluorescence intensity I_{f,o} and reaching a plateau (b). Depletion rate constants (k) were extracted by fitting the experimental data to Equation 6. The normalized depletion rate as a function of distance for the angled geometries (60°, 90°, and 120° NanoEP devices) is also modeled by exponential decay in Equation 6 for determining the characteristic length and Δkₙₒᵣₘ for the rate constant heat maps and the delivery of BSA A647 in the triangle, however rather than a change over time, the change would occur over a distance. Time-lapse imaging data were analyzed using Python 3.11.5 with the packages cv2, matplotlib, seaborn, scipy, and sklearn. Exponential regression models were optimized for each pixel by minimizing the sum of squared errors. The depletion rate constants were visualized as a heat map, where each pixel directly corresponds to the same location in the sample, and the color intensity reflects the value of the depletion rate constant. For the binned calcein depletion data, MATLAB was used to measure the fluorescence intensity decrease over time in each bin.
Edge Effects in NanoEP Device with Membranes in Circular Geometry

To visualize spatial variations in cargo delivery and depletion during NanoEP, a test device fabricated described above comprises indium tin oxide (ITO) transparent electrodes, a polydimethylsiloxane (PDMS) chamber (which contains the cell media or electroporation buffer and defines the active region corresponding to the membrane geometry), and a nanoporous track-etched polycarbonate (PCTE) membrane substrate on which a monolayer of cells were cultured (FIG. 1A, shows two views of the NanoEP device). In conventional NanoEP device designs with circularly shaped active regions of nanopore membranes, a pronounced edge effect is consistently observed, where cargo transport (characterized by its vertical flux) is enhanced at the periphery of the cell monolayer compared to the center, when a perpendicular electric field was applied between the planar ITO electrodes.

This effect was evident in both cargo delivery and depletion contexts using HT-1080 cells, a standard human epithelial fibrosarcoma cells. Cargo of various sizes, from small molecules (molecular weight of approximately 700 Da) to larger plasmids (approximately 5 MDa), exhibited similar trends. Under conventional NanoEP delivery conditions, propidium iodide (molecular weight of approximately 700 Da), a membrane impermeable dye, showed greater cellular uptake at the membrane edge versus the center of the device, as did pLenti3.7-DsRed plasmid (approximate molecular weight of about 5 MDa) stained with YOYO-1 dye. Protein delivery followed the same pattern: cells internalized fluorescently labeled (fluorescent Alexa Fluor™ 488 conjugated Bovine Serum Albumin (BSA-AF488)) with intensities that declined progressively toward the membrane center. Delivery of BSA-AF488 in different cell types including immortalized human corneal epithelial cells (HCE-S), human osteosarcoma cells (U2OS), and immortalized human astrocytes (IM-HA) also showed greater uptake at the membrane edge versus the center of the device. See for example, FIG. 1B. Edge enhanced delivery of BSA-AF488 was also observed in other electroporation buffers, such as media and phosphate-buffered saline (PBS), as well as different electrodes. Depletion studies, where intracellular cargo (e.g., calcein) removal was tracked from HT-1080 cells (e.g., under 15 V, 20 Hz square wave pulses with 200 μs pulse widths for 90 seconds), resulted in a similar edge effect observed in delivery experiments. In a 2 mm diameter circular device, calcein depletion initiated at the periphery and propagated inward, with cells at the edge (FIG. 1C) exhibiting higher depletion rates (k). Because depletion flux measurements decouple cargo concentration spatial heterogeneity from electric field effects, they provide a more direct readout of the field distribution (versus when the NanoEP devices are operated in the delivery mode) and reinforce the notion that electric field non-uniformity, driven by the membrane-edge, governs NanoEP performance.

To investigate the role length scale plays on the observed edge effect, depletion experiments in a larger 6 mm diameter circular device are conducted using 15 V, 20 Hz square wave pulses with 1 ms pulse widths for 120 s (n=3). To accommodate the lower imaging resolution as a result of the lower magnification used to fit the entire 6 mm device in one frame, fluorescence intensity across six concentric radial zones (referred to as radial “bins”) are analyzed to quantify spatial variations, rather than single cells, to assess local fluctuations across the membrane. Fluorescence intensity (I_{f}) in each bin was tracked over time until reaching a plateau (b) and depletion rate constants (k) were extracted by fitting the experimental data to Equation 6. The circular device was radially divided into six concentric radial zones, and fluorescence decay in each bin was tracked over time. A clear gradient emerged from the outermost edge bin (R6) to the innermost center bin (R1), confirming the presence of NanoEP edge effects at larger scales (FIG. 1D). For the circular device geometry, the presence of such edge effects were consistently identified, which resulted in heightened delivery or removal rates near the edge of the devices compared to the center in the lateral x-y plane, parallel to the parallel plate ITO electrodes. Across all circular membrane geometries tested, observed heightened delivery or removal fluxes near the edge of the cell monolayer compared to the center is observed, reflecting lateral non-uniformities parallel to the planar ITO electrodes. Control experiments without applied fields (not shown) confirmed that this edge effect pattern was due to electric-field-driven cargo transport—not photobleaching or buffer exposure.

It is hypothesized that the observed edge effects arise from lateral non-uniformities in the electric field (e.g., perpendicularly focused) across the nanoporous membrane. To model the observed trends in the cargo transport rate as a function of voltage, the simplified Nernst-Planck equation is adapted, neglecting molecular diffusion since the calculated electromotive force driven species flux across the nanopore membrane (greater than that of diffusion) dominates cargo transport (FIGS. 1E-1F). For example, Equation 7 is derived that describes the electrophoretic-driven exponential decay of intracellular calcein concentration, as previously described above, where molecular diffusion is neglected since the calculated electrophoretic species flux across the nanoporous membrane is approximately 1350 times greater than that of diffusion and dominates cargo transport. Under this theoretical framework (Equation 8), the measured rate constant (k_{c}) is provided that scales linearly with electric field strength

$$( \frac{d φ}{dz} ) ,$$

diffusion coefficient (D), effective molecule charge (ze), electropore area (Aₚ), the number of electropores (nₚ), and inversely with cell volume (V) and thermal energy (k_{b}T), where k_{b} is the Boltzmann constant (k_{b}) and T is temperature.

$$C ( t ) = {C}_{0} {e}^{- {k}_{c} t} \qquad ( 7 )$$

$${k}_{c} = \frac{Dze}{{k}_{b} T} \frac{1}{{l}_{eff} ( φ )} \frac{d φ}{dz} \qquad ( 8 )$$

Due to the presence of a complex, nonlinear dependence of Aₚ and nₚ on the local voltage φ⁵, several of these hard-to-estimate parameters were consolidated into a single fitting parameter, l_{eff}, with units of length shown in Equation 9. This l_{eff} term represents an effective transport length for charged intracellular cargo exiting through electropores.

$${l}_{eff} ( φ ) = \frac{V}{{A}_{p} ( φ ) {n}_{p} ( φ )} \qquad ( 9 )$$

Using this model, normalized experimental rate constant (kₙₒᵣₘ) values are compared to model k_{c,norm} predictions across applied voltages (FIG. 1E), using physically relevant parameters. Consistent with prior reports, l_{eff} exhibits a sigmoidal dependence on voltage, and scales linearly with the number of membrane nanopores interfacing with each cell, which itself is a function of the PCTE membrane's nanopore diameter. It is found that an approximate 2-2.5 V voltage differential between the edge and center regions of the cell-membrane interface would give rise to a gradient in cargo flux that coincides with the measured normalized rate constants (1 at the edge versus approximately 0.6 at the center, FIG. 1D). It is therefore hypothesized that the enhanced cargo flux at the membrane perimeter stems from a lateral variation in vertical voltage drop across the membrane nanopores (FIG. 1F). Together, these findings suggest that spatial variation in the electrophoretic driving force, governed by the geometric shape confinement of the nanoporous membrane, could underlie the observed edge effect. This spatial heterogeneity offers a means to create predictable gradients in cargo flux or enhance delivery uniformity by tailoring membrane geometry.

### Electrode-Based Evidence of Radial Electric Field Heterogeneity

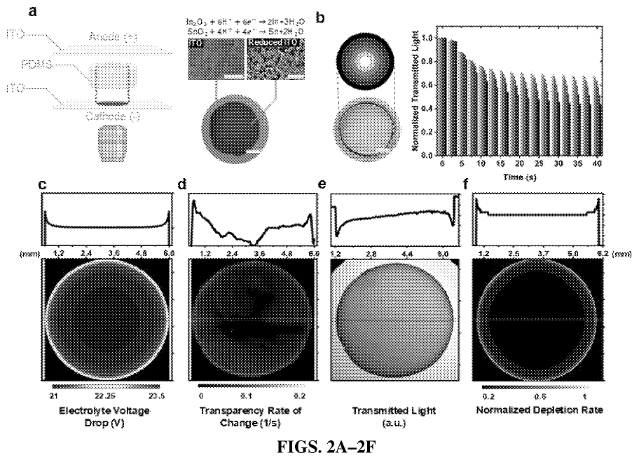

To link the radial heterogeneity in NanoEP flux, both modeling and non-cellular material responses are used to validate that this spatial effect arises from diminished/with anisotropy electric field strength at the device center. A well-characterized electrochemical reaction that induces voltage-dependent changes in electrode optical properties is used, enabling visualization of the lateral field distribution across the device. Specifically, changes in light transmission through the ITO cathodes are measured, which experience reduction currents during NanoEP experiments. FIG. 2A shows the device set up. At the cathode-electrolyte interface (also referred to as the electrode-electrolyte interface or EEI), electrochemical reduction of ITO decomposes its atomic complexes into indium and tin nanoparticles via the reduction reactions:

In₂O₃+6H⁺+6e⁻→2In+3H₂O and Sn₂O₂+4H⁺+4e⁻→Sn+2H₂O,

which substantially decreases optical transparency. Because the rate and extent of the ITO reduction, and the corresponding decrease in optical transparency, are known to scale with the local voltage magnitude (relative to an Ag/AgCl reference or electrochemical overpotential), changes in transmitted light provide a convenient proxy for mapping the lateral electric field distribution across the NanoEP device. To enhance contrast for image analysis, a voltage higher than usual was used. 2 mm devices with PCTE membranes are subjected to 30 V, 20 Hz square-wave pulses (1 ms pulse width) for 40 s (FIG. 2B). Under these conditions, regions of the ITO near the device perimeter consistently darkened more rapidly than central regions, indicating stronger local electric fields at the edge.

The edge effect associated with this electrode reaction persisted in the absence of the PCTE membrane. Using a 6 mm device, the electrolyte voltage drop and the rate and extent of electrode reaction are quantified through both experiments and simulations (FIGS. 2C-2F). Both approaches reveal stronger electric fields near the device perimeter. A 3D finite element (COMSOL) simulation of cylindrical NanoEP devices is performed to map the z-directional voltage drop at each x-y position under a 30 V bias, showing enhanced voltage gradients at device edge compared to the center (FIG. 2C). Experimentally, applying 30 V, 20 Hz square-wave pulses (1 ms pulse width, 60 s duration) produced greater optical transparency changes and faster electrode reactions at the edge, consistent with locally elevated field strengths (FIGS. 2D-2E). Elemental mapping by energy dispersive X-ray spectroscopy (EDX) also confirmed these trends: post-NanoEP EDX maps showed a lower oxygen signal at electrode edges (not shown), consistent with more extensive local ITO reduction. Incorporating the simulated x-y electric field distribution (FIG. 2C) into the modified Nernst-Planck model (Equation 8) predicted higher calcein depletion rate constants at the periphery than the center (FIG. 2F), in agreement with the cargo flux observed experimentally (FIG. 1D).

The emergence of this edge effect is attributed to the interplay between the geometry of both the cell-membrane interface (CMI) and the electrolyte-electrode interface (EEI). Conventional equivalent circuit models of the NanoEP device collapse the x-y dimensions into singular lumped elements from cathode to anode, yielding only one effective voltage drop across the PCTE membrane and cell monolayer. By contrast, finite element simulations of 3D NanoEP devices reveal that when both the EEI extends laterally beyond the CMI interfacial area and when the finite EEI resistance is above a critical threshold (R_{ct}>R_{critical}), the vertical voltage drop is amplified at the lateral device edge, an effect that disappears when the Rot becomes negligible (R_{ct}<R_{critical}). Here, R_{ct} represents a lumped interfacial charge transfer resistance (or EEI impedance) to electron transfer and charge storage, which depends on factors including voltage, buffer composition, electrode material, and electrode area. From these simulations, it is inferred that charge accumulation in electrode regions not directly contacting the electrolyte drives electronic current along the EEI perimeter and ionic current toward the edge of the nanoporous membrane, which represents the paths of least resistance. These combined currents elevate local voltages across the cell-membrane interface, thereby enhancing cargo flux during NanoEP, assuming a laterally uniform vertical transport resistance. While not limiting the present teachings to any particular theory, taken together, these experimental observations (optical+electrochemical) and matching Nernst-Planck model predictions provide compelling evidence that membrane geometry induces lateral (radial) gradients in the electric field during NanoEP. These field gradients, in turn, explain the observed non-uniform NanoEP cargo flux and support the theory that the observed edge effect is controlled by the geometry of both the cell-membrane and electrolyte-electrode interfaces.

Creating Distinct Gradient Profiles with Angled Membrane Geometry

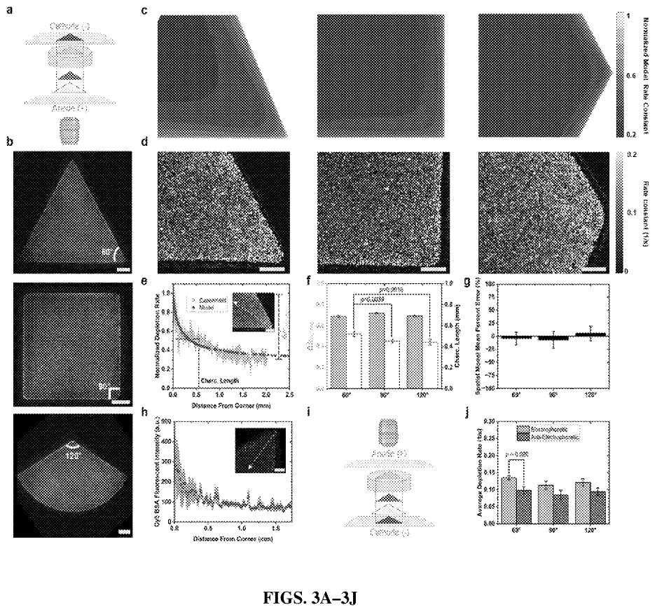

Building on this theoretical framework, how deviations from circular geometries are explored—specifically the introduction of membrane corners (e.g., locations where two edges meet)—affect spatial distribution of cargo flux during NanoEP. It is hypothesized that electric field amplification at corners would generate distinct transport profiles compared to smooth circular boundaries. To test this, membrane geometries are selected with varying internal angles between two adjacent edges (60°, 90°) 120° and compared them to a standard circular device (FIG. 3A).

Calcein depletion experiments were performed under identical square-wave pulsing (15 V, 20 Hz, 1 ms pulse width, 120 s duration) to evaluate the spatial dependence in field-driven molecular transport, with device height and membrane surface area matched to the 6 mm circular control. This ensures comparable impedances between the two electrodes, which is the summed contribution of EEI charge transfer resistance, solution resistance, and membrane ion transport resistance across the nanopores. Depletion was imaged in real time through the anode to avoid optical interference from the cathode reduction (FIG. 3A), and depletion rate constants from the cell monolayers were extracted from the exponential fluorescence decay kinetics for each geometry (FIG. 3B). FIG. 3B shows a triangle-shaped geometry (acute angle of) 60°, a square-shaped geometry (angle of) 90°, and an obtuse circular sector-shaped geometry (angle of) 120°.

Fluorescent micrographs before and after NanoEP for each device geometry were taken (not shown). COMSOL simulations of the x-y electric field distribution were performed for each geometry, and the resulting voltage profiles were fed into the modified Nernst-Planck model to calculate depletion rate patterns (FIG. 3D). The simulations predicted that the sharpest corner (60°) would exhibit the highest local depletion rates, with the highest gradient extending inward from the corner. Experimental calcein depletion data validated these predictions, as shown in representative rate constant heatmaps for each geometry (FIG. 3D).

To quantify lateral gradients in cargo transport, simulated and experimental (n=3) calcein depletion rate constants are compared as a function of distance from membrane corners. The Nernst-Planck model predictions show strong quantitative agreement with experimental results when mapped to the simulated voltage profile for each geometry (FIG. 3E). The model was calibrated using independent datasets collected from corner and center regions, corresponding to the line plot endpoints (FIG. 3E), with training and evaluation data acquired on different days. Calcein depletion rates were measured at each lateral spatial location and fit to an exponential decay function (Equation 6). With rate constants expressed as a function of distance from the corner, a characteristic length is defined (FIG. 3E), the distance at which the change in normalized rate constants (Δkₙₒᵣₘ=k_{corner}−k_{center}) decayed to 1/e of its initial value [(Δkₙₒᵣₘ)/e+k_{center}), where k_{corner} is the depletion rate constant evaluated at the device corner, k_{center} is the asymptote rate constant, and e is Euler's number. Both simulations and experiments revealed that sharper angles produced longer characteristic lengths (FIG. 3F). This trend is intuitive: acute angles (e.g. 60°) keep cells in close proximity to one or more device edges, thereby sustaining an enhanced edge effect over a greater distance from the corner. In contrast, wider angles (e.g. 120°) more closely resemble circular geometries, resulting in a shorter range over which the enhanced electric field persists, hence a shorter characteristic length. This is also compared the Δkₙₒᵣₘ across geometries. While the model predicted larger differences in sharper angles, experimental data showed no significant variation in Δkₙₒᵣₘ between angles. Importantly, simulated rate constants at all lateral positions fell within the 95% confidence interval of experimental values, indicating strong overall agreement (≤12% average error, FIG. 3G). Positive mean percent error

$$( MPE = \frac{100 \%}{{N}_{samples}} {\sum }_{i = 1}^{N} \frac{{y}_{i , measured} - {y}_{i , predicted}}{{y}_{i , measured}} )$$

indicated model underprediction for large-angle geometries, whereas negative MPE values for acute angles (≤90°) suggested overestimation of the lateral electric field enhancement. Overall, these results demonstrate that the model can capture cytoplasmic cargo efflux driven by NanoEP across a range of confined membrane geometries, while also highlighting subtle deviations in how lateral field enhancement scales with the corner angle.

To extend these findings to delivery applications, analogous geometric studies are conducted using Alexa Fluor™ 647-conjugated bovine serum albumin (BSA-AF647) as a model protein cargo. To ensure uniform cargo distribution beneath the nanoporous membrane during NanoEP delivery into the cell monolayer, 200 micrometer (μm)-tall PDMS pillars are incorporated that elevate the membrane from the bottom electrode (FIGS. 6A-6E). For example, first a sticker with pillars is made using Scancut Brother Cutting Machine that is attached to a glass slide. PDMS was spun coat on a glass slide to create a 200 μm layer. The glass slide was then cured overnight at 60 °C, as shown in FIG. 6A. The sticker was removed so only the pillars remained (FIG. 6B). In FIG. 6C, pillars were then plasma bonded or attached to the NanoEP devices with uncured PDMS (followed by an overnight cure at 60 °C). FIG. 6D shows BSA-AF647 localization from folds of the PCTE membrane with no pillars underneath the membrane (scale bar, 1 mm). FIG. 6E shows BSA-AF647 spreading with 200 μm tall pillars underneath membrane (scale bar, 1 mm).

Similar to the depletion experiments, fluorescent intensity of BSA-AF647 was enhanced in cells near the corner of the triangular (60°) devices and decayed nonlinearly toward the center (FIG. 3H). The shape of the normalized BSA-AF647 fluorescence closely matched that of normalized calcein depletion in the same geometry (0.275 mm and 0.345 mm, respectively, for triangular membranes), underscoring the generality of geometry-induced transport gradients across both delivery and depletion modalities. However, their characteristic lengths differed (0.275 mm for BSA delivery versus 0.520 mm for calcein depletion), as did Δkₙₒᵣₘ (0.78 for BSA vs. 0.69 for calcein). The shorter characteristic length and larger Δkₙₒᵣₘ for BSA likely reflect second-order effects of its larger molecular size (67 kDa for BSA vs. 0.6 kDa for calcein), though direct comparison is complicated by differences in how cargo transport was quantified (rate versus amount).

Next, how electrode polarity influenced calcein depletion across geometries is explored. As before, imaging was always performed through the anode to avoid issues associated with cathodic reaction (FIGS. 3A-3I). As expected for a (negatively) charged molecule, calcein exhibited faster average depletion rates in the electrophoretic (anode-downward, FIG. 3A) configuration than when polarity was reversed (anti-electrophoretic, FIGS. 3I, 3J). Importantly, the edge effect persisted in angled devices for both configurations, with corners consistently showing faster depletion than centers. This is attributed to elevated transmembrane potentials (across the CMI) at the edges, which promote more and larger electropores on cells within the applied voltage range. Both the transmembrane potential (governing electropore formation) and the potential across the PCTE membrane (driving cargo flux) contribute to the observed edge effect. Together, these findings validate that the heightened electric field at the edge of the EEI interface accurately translates into predictable spatial flux patterns for both cargo depletion and delivery.

### Increasing Device Perimeter-to-Area Ratio to Enhance NanoEP Delivery Efficiency and Reduce Lateral Variation

After comparing edge effects in cargo delivery and depletion for various angled membrane geometry, a membrane geometry or shape that minimizes lateral variations in NanoEP-mediated delivery while enhancing overall flux, guided by the theoretical model. Conventional NanoEP devices typically employ circular wells, which are convex and characterized by low perimeter-to-area ratios. In such designs, most cells reside far from the membrane edge and thus do not benefit from edge-enhanced field-driven transport, a limitation that becomes more pronounced when scaling up NanoEP systems for larger cell populations.

To address this, in one variation, a new concave “serpentine” membrane geometry (FIG. 4A) with a higher perimeter-to-area ratio (e.g., 2.03) while compared to a perimeter-to-area ratio of 0.67 for a 6 mm circular device of equal area. By positioning more cells in proximity to the membrane edge, it is hypothesized that the serpentine design would amplify the edge effect, thereby boosting intracellular depletion and delivery efficiency while reducing lateral heterogeneity across the cell monolayer.

Experimental calcein depletion rates in serpentine versus 6 mm circular geometries are compared (FIGS. 4A-4B; n=3). The serpentine rate constant profile followed a symmetric U-shaped curve, with a maximum normalized rate difference (Δkₙₒᵣₘ) of approximately 0.47, representing the most uniform depletion observed across all geometries tested. This improvement is attributed to the serpentine's parallel dual-edge configuration, which places more cells near regions of elevated electric field by minimizing the distance of a cell to an edge. By contrast, circular geometries showed a monotonic decrease in rate constants with distance from the edge. Moreover, serpentine devices exhibited higher overall depletion rates, consistent with stronger local electric fields.

Next, BSA-AF647 protein delivery across circular and serpentine devices is assessed (FIG. 4C) using automated cell recognition methods. Population-level fluorescence analysis revealed higher overall intensity in the serpentine geometry compared to the circle (FIG. 4D). Variability in the serpentine device was attributed to local differences in cell adhesion and confluency on the PCTE nanoporous membrane, whereas circular devices displayed a systemic radial gradient of decreasing fluorescence (indicative of amount of cargo delivered) with distance from the edge. Analysis of single-cell fluorescence intensities (FIG. 4D) confirmed a >1.5-fold higher mean fluorescence intensity in serpentine devices compared to circles (5,154 versus 3,319 a.u.). These results demonstrate that employing concave geometries with high perimeter-to-area ratios effectively amplifies edge-enhanced NanoEP flux, thereby improving protein delivery efficiency while minimizing spatial heterogeneity at the device level.

To evaluate edge-enhanced delivery of larger molecular cargos, a plasmid encoding DsRed fluorescent protein (pLenti3.7-DsRed plasmid, about 5 MDa, 100 ng/μL) labelled with YOYO-1 is delivered (FIG. 4E). A PCR purification kit was used to remove unbound YOYO-1, ensuring that virtually all measured fluorescence came from the plasmid-bound dye. Under these conditions, the serpentine NanoEP device geometry produced substantially higher single-cell fluorescent intensity and a greater number of transfected cells than conventional circular nanopore membranes (FIG. 4F). The spatial distribution again exhibited a U-shaped delivery profile, with local fluctuations likely reflecting differences in cell coverage, consistent with trends observed for BSA-AF647 protein delivery. The high plasmid dose used in these experiments facilitated visualization but precluded assessment of downstream protein expression due to plasmid-induced toxicity. Previously, it has been shown that delivery of YOYO-1-labeled plasmid using a 3D NanoEP device produces a two-fold increase in plasmid-associated fluorescence relative to bulk electroporation. Here, an approximately 6-fold increase is observed with the serpentine geometry relative to standard circular NanoEP devices. These results further highlight the advantages of localized electroporation over bulk electroporation and show that membrane geometry is itself a key determinant of delivery performance.

To assess functional delivery, the pLenti3.7-DsRed plasmid delivery experiments are repeated at a lower plasmid concentration (5 ng/μL) and quantified DsRed expression 48 hours post-transfection (FIG. 4G). Single-cell fluorescent analysis confirmed both a higher fraction of DsRed-positive cells and greater mean expression levels in the serpentine devices (FIG. 4H). At this lower dose, cell viability remained >95% after 48 hours and was comparable to untreated controls (FIG. 4K). By contrast, at 100 ng/μL, cells began detaching from the nanoporous membrane 24 hours post transfection, so viability at the higher dose was taken at 24 hours rather than 48 hours to minimize the artifacts from cell loss.

Aggregated data across different cargos show a consistent trend of higher delivery or depletion in the serpentine compared to the circular devices (FIGS. 4I-4J): approximately 2-fold higher calcein depletion rates, approximately 1.5-fold higher BSA-AF647 fluorescent protein delivery, approximately 6-fold higher plasmid delivery, and approximately 1.2-fold higher DsRed expression. These findings further support that devices employing concave membrane shapes with higher perimeter-to-area ratios substantially improve NanoEP-mediated macromolecular transport efficiency and spatial uniformity.

Interestingly, device geometry also altered the distribution of cargo delivered across the cell population. In the circular devices, single-cell fluorescence intensities for all cargos followed the near-normal distributions expected for a largely stochastic delivery process (FIGS. 4D-4H). By contrast, the serpentine geometry produced a log-normal distribution for BSA-AF647 (FIG. 4D) and a broader, positive-skewed bimodal distribution for plasmid delivery and the resulting DsRed expression (FIGS. 4F-4H). This shift is attributed to the emergence of geometric confinement effects in the serpentine design. In the 6 mm diameter circular geometries, most cells reside relatively far from an edge and therefore experience a weaker, more spatially uniform electric field, such that cargo partitioning among neighboring cells is governed primarily by thermal fluctuations and diffusion. In the serpentine devices, however, a much larger fraction of cells lies close to the nanopore membrane perimeter, where the electric field is strongly amplified in a nonlinear manner (FIG. 4B), biasing transport toward edge-proximal cells and broadening the resulting delivery distribution.

The extent and form of this skew also depended on cargo properties. BSA is a relatively small, modestly charged, globular protein with a hydrodynamic radius of only a few nanometers, and was delivered at a substantially higher concentration than the plasmid (2.5 mg/mL vs. 0.1 mg/mL). As a result, although the nonlinear edge field shifts the distribution away from normality, the large number of transported BSA molecules and their lower steric constraints preserve a single observable peak, yielding a skewed but unimodal distribution (FIG. 4D). In contrast, the plasmid is a much larger cargo (approximately 5 MDa) with a high negative charge density, making its transport more sensitive to the nonlinear electric field and more susceptible to low-number fluctuations in nanopore entry and subsequent uptake. It is therefore inferred that the strong positive skew reflects field-enhanced transport of a subset of cells, whereas the bimodality arises from the comparatively low probability of successful plasmid translocation and delivery at the single-cell level (FIG. 4F). Notably, this heterogeneity is also reflected in the downstream DsRed expression profile (FIG. 4H), although with a narrower spread relative to the median intensity. These results demonstrate that increasing the perimeter-to-area ratio is an effective strategy to leverage edge effect amplification of NanoEP cargo flux, enhancing delivery efficiency and reducing lateral heterogeneity across the device.

### Investigating the Effects of Geometric Confinement Across Cell Types and Length Scales

To assess whether benefits of serpentine geometries extend beyond HT-1080 cells, first cargo depletion kinetics in murine NIH/3T3 cells engineered are examined to constitutively express cytosolic green fluorescent protein (GFP). The NIH/3T3 cells were treated using the same electrical conditions applied to calcein-stained HT-1080 cells (15 V, 20 Hz, 1 ms pulse width, 120 s). Under these conditions, the serpentine geometry produced an approximate 1.3-fold increase in GFP depletion rate relative to the state-of-the-art circularly shaped NanoEP device (FIGS. 7A-7B). Despite substantial differences in both cargo size (calcein versus GFP) and cell type, the results indicate that the field-enhancing effects of geometric confinement similarly modulate transport flux.

Next, whether this enhancement generalizes across additional adherent cell types is evaluated by delivering BSA-AF488 to human epithelial breast cancer cells (MDA-MB-231), immortalized human corneal epithelial cells (HCE-S), and immortalized human astrocytes (IM-HA). All cell types were treated with the same delivery conditions used for HT-1080 cells (20 V, 20 Hz, 1 ms pulse width, 10 s). Across all three cell types, the serpentine geometry consistently produced higher cargo delivery than the circular geometry (FIGS. 7C-7D). The magnitude of enhancement varied across cell types, likely reflecting differences in adhesion, spreading, and proliferation behavior, which, as reported previously and reconfirmed here, are key determinants of NanoEP performance. Accordingly, geometric confinement produces the most predictable outcomes under the conditions that promote strong cell adhesion, well-spread morphologies, and monolayer growth.

Beyond generalizability across cell types, practical translation of NanoEP also requires that these geometric effects persist at larger device scales relevant to tissue engineering and cell manufacturing. To examine the effect of transfection throughput, BSA-AF488 is delivered to HT-1080 cells using circular devices of increasing diameter (FIG. 7E), with a square wave at 20 V, 20 Hz, 1 ms pulse width that is applied for 10 s. Averaged single-cell fluorescence measurements (n=3 devices for each condition) showed a monotonic decrease in cargo delivery with increasing device diameter (FIG. 7F). Conventional well-plate-equivalent dimensions, which are attractive for integration into scalable manufacturing workflows, are indicated along the x-axis (FIG. 7F). This shows that as device surface areas scale up to meet manufacturing throughput, maintaining adequate transport flux would require higher applied voltages, which could in turn promote undesired electrochemical reactions or increase cellular stress. However, this limitation could be circumvented by designing the membrane geometry to focus the electric field over larger areas. Therefore, circular and serpentine devices are matched at 1 time (1×), 2 times (2×), and 5 times (5×) surface areas, where 1 time corresponds to the area of the 6 mm diameter circular device (FIGS. 7E, 7G). Using the same BSA-AF488 delivery conditions (20 V, 20 Hz, 1 ms pulse width square wave, 10 s) a progressive decline in average cellular fluorescence is observed with increasing area in circular devices (FIG. 7H). In contrast, serpentine devices maintained comparable delivery levels across all tested surface areas (FIG. 7H). When comparing the 5× serpentine device to the 5× circle device, there was a 1.52 fold increase in average intensity per cell. Delivery efficiency of the 5× serpentine device is compared to circular devices. In addition to a decrease in fluorescence intensity as a function of circle diameter, a decrease in delivery efficiency is also observed (FIGS. 7I, 7J). The 5× serpentine device had comparable intensities and efficiencies to the 2 mm-6 mm diameter circular devices while increasing the cell throughput by 5-45 fold. However, when compared to the 13.4 mm diameter circular device (5× circle), the 5× serpentine had a 1.8 fold increase in delivery efficiency of 41% to 73%. These results indicate that conventional circular geometry becomes increasingly suboptimal upon scale-up, whereas geometries that localize and concentrate the electric field can preserve delivery performance over larger areas. Taken together, these findings establish geometric confinement as a generalizable strategy to improve NanoEP-mediated cargo depletion and delivery across multiple cell types and across device length scales relevant to higher-throughput applications.

### Understanding the Role of Membrane Geometry in Electric Field Distribution and Cargo Transport Control

Through combined experimental and simulation-based investigation, it is demonstrated that lateral nanoporous membrane geometry directly influences vertical NanoEP flux (along the z-axis) by modulating the in-plane (x-y) electric field distribution across the membrane nanopores. By controlling this lateral field distribution, the spatial uniformity of intracellular cargo delivery and depletion can be modulated across a cell monolayer. Using a modified Nernst-Planck molecular flux model, cargo transport rates (e.g., depletion kinetics) are accurately predicted from device-specific lateral voltage profiles by introducing an effective transport length (l_{eff}), defined as the ratio of cell volume to total electropore area, which is a function of local electric fields. Conceptually, l_{eff} represents an ensemble-averaged path length for cargo molecules leaving the cell: smaller l_{eff} values correspond to faster depletion rates. This modeling framework enables rational design of membrane geometries to achieve targeted intracellular delivery or depletion profiles.

It was found that l_{eff} must be tuned for different sub-regions of the CMI (cell-membrane interface) to accurately model cargo transport. Subdividing the nanoporous membrane into lateral “corner” and “center” zones substantially improved model fidelity. As expected from the sigmoidal dependence of l_{eff} on local voltage (φ), which reflects the voltage-driven increase in the electropore number (nₚ) and area (Aₚ) in cells during NanoEP, l_{eff} was lowest at device corners. Specifically, l_{eff} values were calculated of 2.33 mm (center) and 0.92 mm (corner), confirming substantially enhanced field-driven cargo transport near corners of the nanoporous membrane. While these values exceed the dimension of a single cell, they are empirical measures that are likely to include transport retardation effects from thermal fluctuation (molecular random walk) and electropore cycling (opening/closing). Cells were pulsed with a 2-4% duty cycle at 20 Hz (50 ms period), and prior studies suggest large electropores close within approximately 100 μs. Thus, because

$${lim}_{{A}_{p} {n}_{p} \to 0} {l}_{eff} = ∞ ,$$

and the field is “off” more than “on”, measured l_{eff} values are higher than physically intuitive. Using the expression

$${l}_{eff} = \frac{V}{{A}_{p} ( φ ) {n}_{p} ( φ )} ,$$

with an estimated HT-1080 cell volume V=2×10⁻¹⁵ m³, electropore radius rₚ=15 nm (Aₚ=706 nm²), 1212-3065 electropores per cell are estimated, with the highest density near corners. Given approximately 3500 nanopores underneath each cell (nanopore diameter=200 nm), these estimates suggest 35-88% of the nanopores within the PCTE membrane actively contribute to intracellular cargo transport in a confluent cell monolayer, assuming one cell electropore per membrane nanopore. In reality, a single nanopore may host multiple electropores of varying radii, so the actual number of employed nanopores will be less than calculated.

Optimizing plasmid delivery via NanoEP has been a major focus and presents unique challenges as compared to protein delivery. For example, successful gene expression requires uptake and an optimal intracellular copy number, enough to enable detectable expression without inducing toxic overexpression. Here, membrane geometry, an often-overlooked aspect of NanoEP device design, is shown to profoundly influence both delivery and expression outcomes, introducing a hidden layer of variability unless carefully controlled. The analysis of this design framework suggests substantial room for improvement over current electroporation platforms, with a potential for approximately 12-fold enhancement to bulk electroporation and 6-fold enhancement relative to conventional NanoEP for large plasmid delivery. Importantly, this augmented delivery efficiency in serpentine geometry devices is maintained as cell throughput and device size increases, outperforming other state-of-the-art substrate-based transfection methods (FIG. 7J) by achieving efficacious delivery (>70%) at cell throughputs previously unseen in literature (>1.5×10⁵).

These findings underscore the importance of substrate geometric engineering that improves delivery efficiency and/or uniformity alongside increased transfection throughput. The serpentine geometry highlights the impact of edge-enhancing device structures. In the serpentine designs contemplated by certain variations of the present disclosure, cells are positioned adjacent to an “edge,” which acts as a high-resistance barrier to the electric field and mass transport. This boundary concentrates the electric field and flux in nearby regions of lower resistance, thereby amplifying localized cargo transport. Thus, the present disclosure contemplates a generalizable design principle: nanoporous membranes can be patterned to strategically increase edge-adjacent areas and thereby enhance transfection efficiency. Further, identifying the optimal void fraction and feature size of patterned PCTE substrates can facilitate uniform NanoEP transfection across large contiguous areas (e.g., T25 flask-scale formats).

Although the electric field is the dominant factor shaping cargo delivery profiles, additional factors not captured by the COMSOL simulations or modified Nernst-Planck model may also contribute. For instance, electroosmotic flow can impose pressures up to about 1 kPa on the cell monolayer during electroporation, which may oppose the migration of negatively charged cargo (e.g., calcein) toward the anode (+), particularly near the device center. Additional sources of system-level variability include the stochastic nanopore distribution in PCTE membranes, as well as non-uniform cell coverage and membrane contact (CMI), each of which can introduce noise and heterogeneity in cargo flux. Despite these complexities, lateral electric field distributions, and thus intracellular delivery or depletion outcomes in NanoEP, can be predicted and programmed through membrane geometry alone. Breaking lateral symmetry enables device architectures that promote either more uniform or intentionally patterned delivery. This form of geometric control provides a practical route toward scalable, substrate-based transfection platforms for clinically relevant applications, including complex 2D and potentially 3D tissue models. It is further contemplated that increasing the perimeter-to-area ratio appears to be a promising strategy for expanding effective substrate surface area while enhancing delivery performance.

In this example, it is demonstrated that nanopore membrane geometry plays a significant role in shaping the in-plane (x-y) electric field distribution and, consequently, drives anisotropic molecular flux during NanoEP-mediated cargo delivery or removal. By systematically varying nanoporous membrane geometries, the spatial heterogeneity of cargo transport can be predictably controlled through lateral electric field manipulation. A geometry-dependent edge effect is identified and characterized, where enhanced cargo transport occurs near the perimeter of the nanoporous membrane due to lateral gradients of the vertically amplified electric field. By coupling experimental results with finite element voltage simulations and a modified Nernst-Planck flux model, strong agreement between predicted and observed lateral heterogeneity in cargo flux is established, validating the underlying electrokinetic mechanism. Building on this insight, device geometries are introduced with various internal angles to guide spatial gradients in both cargo delivery and depletion. Concave device geometries with high perimeter-to-area ratios are demonstrated to significantly reduce the average distance between target cells and membrane edges, resulting in more efficient and more uniform delivery of protein and plasmid cargos across multiple cell types than state-of-the-art NanoEP device designs, thereby providing a practical path toward scale-up. Overall, lateral membrane geometry serves as an important design parameter for controlling localized electric fields and the resulting intercellular cargo distribution in substrate-based delivery platforms.

Thus, in certain aspects, the present disclosure provides that NanoEP membrane geometry affects the cell manipulation device's x-y electric field distribution and, ultimately, causes anisotropic flux during cargo delivery or removal. Membrane geometry is leveraged to control the distribution of the electric field in NanoEP systems, thus controlling the degree of heterogeneity of cargo delivery and removal in a cell population. The present disclosure is believed to provide the first systematic examination of how membrane geometry affects the uniformity of cargo delivery and depletion in NanoEP systems. The existence of an edge effect phenomenon in NanoEP systems is identified and characterized, where higher delivery and depletion of cargo along the perimeter of different geometries is observed, establishing that the phenomenon is likely due to non-uniform electric fields. These experimental results are connected to theory using finite element method (FEM) electric field simulations as inputs for a Nernst-Planck model of cargo motion during NanoEP, and good agreement is found to demonstrate the predictably of spatial heterogeneity in cargo removal. Using the knowledge on edge effects in NanoEP systems, shapes with various internal angles are introduced to create prescribed gradients of cargo delivery and depletion. For example, by changing the membrane geometry's angles (60°, 90°) 120°, unique cell populations of prescribed gradients can be created. Additionally, improved uniformity is demonstrated by minimizing the distance of a cell to an edge by increasing the perimeter-area ratio of the membrane geometry. By increasing the perimeter-area ratio to minimize the average distance of a cell to an edge, a more global uniform cell population is created. By controlling the x-y distribution through leveraging membrane geometry, substrate-based transfection methods now have the potential to be highly scalable models for complex 2D tissues. Further, by leveraging membrane geometry in NanoEP systems, a non-viral based method can create varying degrees of heterogeneity in cell populations.

Thus, in certain aspects, the present disclosure provides that NanoEP membrane geometry affects the cell manipulation device's x-y electric field distribution and, ultimately, causes anisotropic flux during cargo delivery or removal. Membrane geometry is leveraged to control the distribution of the electric field in NanoEP systems, thus controlling the degree of heterogeneity of cargo delivery and removal in a cell population. After what is believed to be the first systematic examination of how membrane geometry affects the uniformity of cargo delivery and depletion in NanoEP systems, the existence of an edge effect phenomenon in NanoEP systems is identified and characterized, where higher delivery and depletion of cargo along the perimeter of different geometries is observed. This establishes that the phenomenon is likely due to non-uniform electric fields. These experimental results are connected to theory using finite element method (FEM) electric field simulations as inputs for a Nernst-Planck model of cargo motion during NanoEP, and good agreement is found to demonstrate the predictably of spatial heterogeneity in cargo removal. Using the knowledge on edge effects in NanoEP systems, shapes with various internal angles are introduced to create prescribed gradients of cargo delivery and depletion. For example, by changing the membrane geometry's angles (60°, 90°, 120°), unique cell populations of prescribed gradients can be created. Additionally, improved uniformity is demonstrated by minimizing the distance of a cell to an edge by increasing the perimeter-area ratio of the membrane geometry. By increasing the perimeter-area ratio to minimize the average distance of a cell to an edge, a more global uniform cell population is created. By controlling the x-y distribution through leveraging membrane geometry, substrate-based transfection methods now have the potential to be highly scalable models for complex 2D tissues. Further, by leveraging membrane geometry in NanoEP systems, a non-viral based method can create varying degrees of heterogeneity in cell populations.

### Example 2

The redox active buffer fluid composition: MilliQ water (Millipore sigma) was used as the solvent or carrier of the buffer/electroporation fluid composition. The base components are 5-15 mM potassium phosphate buffer, 10-20 mM 4-(2-Hydroxyethyl) piperazine-1-ethane-sulfonic acid (HEPES) buffer, 125-200 mM sugar (e.g., sucrose, trehalose, etc.). Finally, 1-10 mM of potassium ferrocyanide and 1-10 mM of potassium ferricyanide are added for the redox active component. An additional redox active component that can supplement or complement the ferrocyanide is 1-50 mM ferrocenemethanol. Optional components include 1-5 mM Adenosine triphosphate (ATP) and 0.1-1 v % Dimethyl sulfoxide (DMSO). The pH range is 7-7.5, with pH adjustments using potassium hydroxide or hydrochloric acid as needed. The conductivity is in the range 0.1-0.5 S/m. All chemicals were purchased from Sigma Aldrich.

FIG. 10 shows a simulation of NanoEP device geometry, comparing the electrolyte voltage drop (electromotive driving force) over different charge transfer resistances and cell configurations. The cells either cover the nanochannel membrane (PCTE) completely (left), or there was unequal cell coverage with a gap in the middle (right). In both cases, the simulations with a lower charge transfer resistance (R_{CT}) had a higher electromotive driving force in the liquid. Liquid conductivity was set to 0.2 S/m.

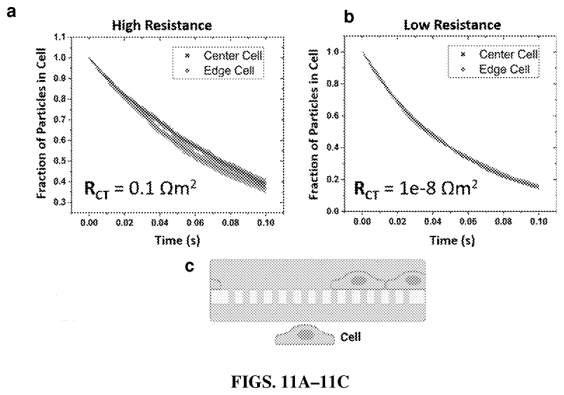

FIGS. 11A-11C show simulation geometry from FIG. 10 (on the right), where transport rates of charged molecules out of cells on a PCTE membrane are simulated. The “Center Cells” were located at radius=0 mm and the “Edge Cells” were located at radius ≅1 mm. These results indicate that having a high RCT results in a lower driving force over the liquid and a slower and more variable rate of cargo transport out of the cell. Decreasing RCT raises the electromotive force in the liquid and results in faster and more consistent cargo flux.

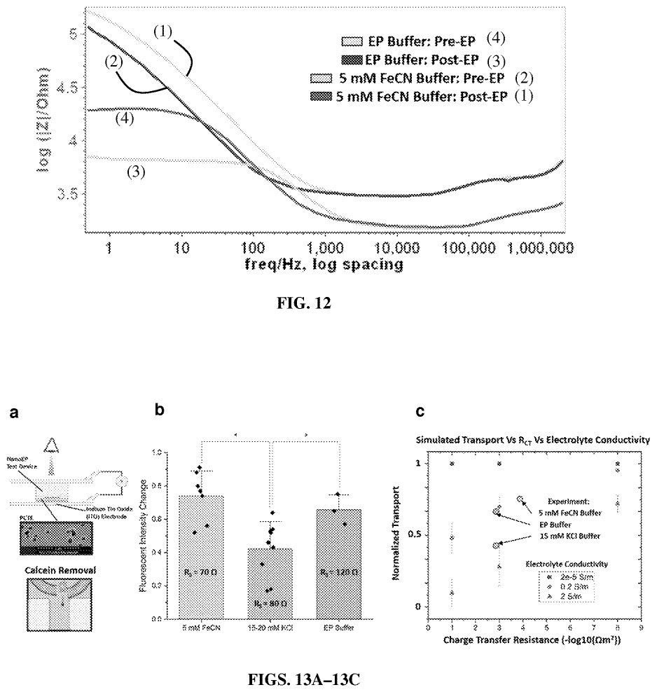

FIG. 12 shows experimental impedance data collected for BioRad's commercial electroporation buffer (EP Buffer) and electroporation buffer with equimolar potassium ferrocyanide and potassium ferricyanide (FeCN) designated FeCN Buffer. Low frequency (<100 Hz) impedance data is associated with the charge transfer at the electrode interface. The commercial, non-redox EP buffer has a pre-electroporation impedance that is greater than 10 times (>10×) higher at its peak than the impedance of the buffer containing FeCN prepared according to certain aspects of the present teachings. After electroporation (EP), the commercial EP buffer's impedance decreases, which is likely associated with reconfiguration of the electrode interface used in this experiment. The FeCN buffer, however, saw an impedance increase after EP. This is associated with the reaction of some FeCN at the electrode interface, a concentration gradient develops, and the impedance increases because there is less FeCN to react with.

FIGS. 13A-13C show experimental electroporation data collected using the test device shown in FIG. 13A A cell cytoplasm stain, calcein, is used as a fluorescent cargo to remove from cells using NanoEP. From the fluorescent intensity change data in FIG. 13B, the 5 mM FeCN buffer and the commercial EP buffer had comparably high overall transport (>60%); however, the commercial EP buffer with 15 mM potassium chloride had a significantly lower (p<0.05) overall transport. This is surprising, as the conductivity of the potassium chloride (KCl) and FeCN buffers are similar (σ≅0.45 S/m), yet the FeCN buffer had almost 2 times the transport, on average. Electroporation conditions were at 16 V, 20 Hz, 1 ms square pulses applied for 20 s. In FIG. 13C, the expected transport out of the cells as a function of the charge transfer resistance (R_{CT}) and buffer conductivity was simulated, and found the experimental results align well with the simulated predictions. The commercial EP buffer and the buffer with KCl have the same R_{CT}, but the KCl buffer is more conductive and thus has lower transport. Notably, the FeCN buffer is also more conductive, but has a lower RCT and experimentally an increase in flux is observed. The simulations predict that flux increases as R_{CT} decreases, and thus the simulation trends and experimental data are in agreement.

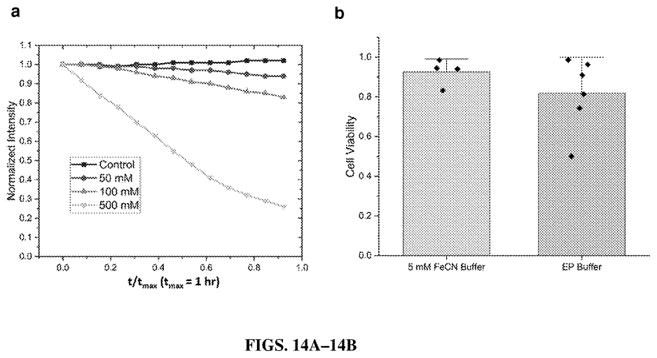

FIG. 14A shows HT-1080 (human fibrosarcoma) cell viability data collected using an automated imaging microscope with a cell incubator (37 °C, 5% CO₂). The fluorescent intensity is reported using calcein as a cell cytoplasm marker, and should indicate their viability as it will only leak out if holes in their membrane are opened. No electroporation was done in this experiment, the cells were simply incubated in different concentrations of FeCN buffer, with the control being in complete Eagle's minimum essential media (CEMEM) cell growth solution. This result indicates that the cell membrane is only compromised at FeCN concentrations of greater than about 100 mM, as electroporation experiments typically take less than about 20 minutes. In FIG. 14B, HT-1080 cell viability data is collected 24 hours post-electroporation. Experimental conditions of 16 V, 20 Hz, 1 ms square pulses for 20 s. No significant difference was found between the commercial EP buffer and the 5 mM FeCN buffer, and both viabilities were reasonably high on average (greater than about 80%). Dead cells were counted by comparing the total number of cells to the number of cells with their nucleus stained red from ethidium homodimer (dead cell stain).

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A cell manipulation device comprising:
a first electrode;
a second electrode;
an insulating porous membrane configured to support a plurality of cells, wherein the insulating porous membrane comprises a plurality of pores and the insulating porous membrane defines an active region having a cross-sectional shape having a perimeter-to-area ratio of greater than or equal to about 0.75; and
at least one cargo material to be delivered to at least a portion of the plurality of cells disposed on the insulating porous membrane through at least one pore of the plurality of pores in the active region when a potential is applied to the first electrode and the second electrode.

2. The cell manipulation device of claim 1, further comprising a receptacle disposed adjacent to the insulating porous membrane that is electrically non-conductive and configured to receive an electroporation fluid comprising the at least one cargo material.

3. The cell manipulation device of claim 2, wherein the receptacle defines an opening having the cross-sectional shape and defining the active region through which the electroporation fluid comprising the at least one cargo material can pass.

4. The cell manipulation device of claim 1, wherein the perimeter-to-area ratio is greater than or equal to about 2.

5. The cell manipulation device of claim 1, wherein the cross-sectional shape defines at least two sides.

6. The cell manipulation device of claim 3, wherein the cross-sectional shape is selected from the group consisting of: a polygon, a triangle, a rectangle, a square, a quadrilateral, a pentagon, a hexagon, a serpentine shape, and combinations thereof.

7. The cell manipulation device of claim 1, wherein the cross-sectional shape comprises a serpentine shape.

8. (canceled)

9. The cell manipulation device of claim 1, wherein the at least one cargo material comprises a biomaterial selected from the group consisting of: a pharmaceutical active ingredient, a protein, a peptide, a growth factor, a biofactor, a cytokine, a hormone, a gene therapy material, a cell therapy material, a nanomaterial, an imaging agent, and combinations thereof.

10. The cell manipulation device of claim 9, wherein the biomaterial comprises a genetic therapy agent comprising a polynucleotide agent selected from the group consisting of: DNA, RNA, plasmids, plasmid DNA (pDNA), short interfering sequence of double stranded RNA (siRNA), messenger RNA (mRNA), transfer RNA, ribosomal RNA, small nuclear RNA, single stranded DNA, microDNA, guide RNA, CRISPR CAS-9, aptamers, and combinations thereof.

11. The cell manipulation device of claim 1, wherein the insulating porous membrane comprises polycarbonate.

12. A cell manipulation device comprising:
a first electrode;
a second electrode;
an insulating porous membrane having a surface configured to support a plurality of cells, wherein the insulating porous membrane comprises a plurality of pores and the insulating porous membrane defines a pattern of electrically non-conductive material disposed on the surface to define an active region; and
at least one cargo material to be delivered to at least a portion of the plurality of cells disposed on the insulating porous membrane through at least one pore of the plurality of pores in the active region when a potential is applied to the first electrode and the second electrode.

13. The cell manipulation device of claim 12, wherein the electrically non-conductive material comprises a polymeric material selected from the group consisting of: polydimethylsiloxane (PDMS), an epoxy-based photoresist, and combinations thereof.

14. The cell manipulation device of claim 12, wherein the pattern defines an array comprising a plurality of shapes selected from the group consisting of: a dot, a circle, a polygon, a triangle, a rectangle, a square, a quadrilateral, a pentagon, a hexagon, a serpentine shape, and combinations thereof.

15-42. (canceled)

43. An electroporation fluid composition for an electroporation process for at least one cell, the electroporation fluid composition comprising:
a redox active compound;
at least one cargo material to be delivered to or removed from the at least one cell when a potential is applied to the electroporation fluid composition; and
a carrier liquid.

44. The electroporation fluid composition of claim 43, wherein the redox active compound comprises potassium ferrocyanide, potassium ferricyanide, ferrocenemethanol, and combinations thereof.

45. The electroporation fluid composition of claim 43, wherein the redox active compound comprises potassium ferrocyanide and potassium ferricyanide.

46. (canceled)

47. The electroporation fluid composition of claim 46, wherein the redox active compound further comprises ferrocenemethanol.

48. (canceled)

49. The electroporation fluid composition of claim 43, wherein a total amount of the redox active compound is present at a concentration of greater than or equal to about 1 mM to less than or equal to about 70 mM in the electroporation fluid composition.

50. (canceled)

51. The electroporation fluid composition of claim 43 further comprising at least one component selected from the group consisting of: a salt, a sugar, a buffering agent, a chelating agent, a pH adjusting agent, adenosine triphosphate (ATP), and combinations thereof.

52. The electroporation fluid composition of claim 43, wherein the at least one cargo material comprises a biomaterial selected from the group consisting of: a pharmaceutical active ingredient, a protein, a peptide, a growth factor, a biofactor, a cytokine, a hormone, a gene therapy material, a cell therapy material, a nanomaterial, an imaging agent, and combinations thereof.

53-64. (canceled)
